(19) United States
(12) Patent Application Publication
(10) Pub. No.: US 2009/0175866 A1
(43) Pub. Date: Jul. 9, 2009

(54) TREATMENT OF B-CELL MALIGNANCIES

(76) Inventors: Avner Yayon, Sitria (IL); Eran Rom, Rehovot (IL); Suzanne Trudel, Toronto (CA)

Correspondence Address: WINSTON & STRAWN LLP;PATENT DEPARTMENT, 1700 K STREET, N.W., WASHINGTON, DC, 20006, US

(21) Appl. No.: 11/718,530

(22) PCT Filed: Nov. 3, 2005
(86) PCT No.: PCT/IL05/01154

§ 371 Date: Feb. 27, 2009

Related U.S. Application Data
(60) Provisional application No. 60/624,549, filed on Nov. 4, 2004.

(51) Int. Cl. A61K 39/395

(52) U.S. Cl. 424/135.1; 424/174.1; 424/144.1; 424/133.1

(57) ABSTRACT

The present invention generally relates to a pharmaceutical composition and to an improved method of preventing, attenuating and treating multiple myeloma by administering to an individual in need thereof at least one antibody to fibroblast growth factor receptor 3 (FGFR3). In particular, the at least one FGR3 antibody induces apoptosis of myeloma cells expressing wild type FGFR3.

### FIELD OF THE INVENTION

The present invention relates to a pharmaceutical composition and a method of preventing, attenuating and treating B-cell malignancies, in particular multiple myeloma (MM), by administering to an individual in need thereof at least one antibody to fibroblast growth factor receptor 3 (FGFR3). In particular, the at least one FGFR3 antibody induces apoptosis of myeloma cells expressing wild type FGFR3.

### BACKGROUND OF THE INVENTION

### Fibroblast Growth Factors

Fibroblast Growth Factors (FGFs) constitute a family of over twenty structurally related polypeptides that are developmentally regulated and expressed in a wide variety of tissues. FGFs stimulate proliferation, cell migration and differentiation and play a major role in skeletal and limb development, wound healing, tissue repair, hematopoiesis, angiogenesis, and tumorigenesis (reviewed in Ornitz and Itoh, Genome Biology 2001, 2 (3): reviews 3005.1-3005.12).

The biological action of FGFs is mediated by specific cell surface receptors belonging to the receptor protein tyrosine kinase (RPTK) family of protein kinases. These proteins consist of an extracellular ligand binding domain, a single transmembrane domain and an intracellular tyrosine kinase domain that undergoes phosphorylation upon binding of FGF. The FGF receptor (FGFR) extracellular region contains three immunoglobulin-like (Ig-like) loops or domains (D1, D2 and D3), an acidic box, and a heparin-binding domain. Four FGFR genes encoding for multiple receptor variants have been identified to date.

B-Cell Associated Malignancies

B cell neoplasms include precursor B-lymphoblastic leukemia/lymphoma (precursor B-cell acute lymphoblastic leukemia), B-cell chronic lymphocytic leukemia/small lymphocytic lymphoma, B-cell prolymphocytic leukemia, Lymphoplasmacytic lymphoma, Splenic marginal zone B-cell lymphoma, Hairy cell leukemia, Plasma cell myeloma/plasmacytoma, Extranodal marginal zone B-cell lymphoma of MALT type, Nodal marginal zone B-cell lymphoma, Follicular lymphoma, Mantle-cell lymphoma Diffuse large B-cell lymphoma, Monocytoid B-cell lymphoma and Multiple myeloma.

Multiple Myeloma

Multiple myeloma (MM) is a fatal hematopoietic malignancy of plasma cells. Plasma cells that undergo IgH switch recombination typically home to the bone marrow, where they reside. Interaction with bone marrow stroma leads to proliferation of malignant plasma cells and tumor formation. Progression of intramedullary myeloma is associated with increasingly severe secondary features that include lytic bone disease and osteoporosis, hypercalcemia, anemia, immunodeficiency and renal impairment.

Multiple myeloma is the second most prevalent blood cancer after non-Hodgkin's lymphoma. It represents approximately 1% of all cancers and 2% of all cancer deaths. Although the peak age of onset of multiple myeloma is 65 to 70 years of age, recent statistics indicate both increasing incidence and earlier age of onset.

For decades, MM treatment has been based on cytotoxic chemotherapy, primarily standard-dose oral melphalan combined with prednisone. High-dose melphalan therapy combined with autologous bone marrow transplantation to reduce myelotoxicity (Child et al. (2003) NEJM 348; 19 1875-1883) has also been evaluated and results in a modest increase in overall survival over standard dose chemotherapy.

Several genetic determinants have been shown to be responsible for the onset and progression of MM. Approximately 15%-20% of the MM cases are associated with a chromosomal translocation, t(4; 14)(p16.3; q32), that deregulates the expression of MMSET from der (4) and FGFR3 from der(14). In particular, wild type FGFR3 becomes ectopically expressed at very high levels and induces proliferative signals in myeloma cells. This translocation has been shown to be a primary event in MM and in some cases activating mutations of FGFR3 are acquired as the disease progresses. Recent studies demonstrate that patients with t(4; 14) have a particularly poor prognosis.

FGFR3 has been validated by in vitro and in vivo animal studies as a therapeutic target for MM. In principle, an ideal FGFR3 inhibitor useful for the treatment of MM will exhibit the following properties:

Recognize FGFR3 and be able to inhibit the activated forms of wild type and mutated FGFR3.

FGFR3 specific, i.e. does not inhibit other FGFR or tyrosine kinase proteins.

Biocompatible; i.e. non-immunogenic and non-toxic to the patient.

Long half-life in blood stream.

International patent application publication WO 02/102973, co-assigned to some of the assignees of the present invention, discloses antibodies to receptor tyrosine kinases, specifically anti-Fibroblast Growth Factor Receptor 3 (FGFR3) antibodies. Certain antibodies shown to be specific for FGFR3 neutralize FGFR3 activity and are useful for treating skeletal dysplasias such as achondroplasia and proliferative diseases such as bladder cancer. That disclosure notes a list of proliferative diseases in which FGF receptors are known to be involved including inter alia multiple myeloma.

International patent application publication WO 03/004056 teaches a method of treating multiple myeloma using a K121-like antibody that induces apoptosis in myeloma cells.

PEGylation has been employed to modify antibodies, both single chain and monoclonal to achieve greater solubility and longer circulating life in vivo. PEG (40,000) was conjugated to the mabs, N12 and L26, specific to the ErbB2 (HER2) oncoprotein (Hurwitz et al. (2000) Cancer Immunol. Immunother. 49 226-234). Koumenis et al. (Int. J. Pharmaceut. 198 83-95 (2000)) also achieved an increase in the circulation half life of the F(ab′)2 form of a humanized anti IL-8 by PEG conjugation.

Traditional methods of treating B-cell malignancies, including chemotherapy and radiotherapy, have limited utility due to toxic side effects. The use of monoclonal antibodies restricts their toxicity to cells expressing the target antigen. The art has not yet identified an effective anti-FGFR3 antibody for the prevention or treatment of multiple myeloma.

Citation of any document herein is not intended as an admission that such document is pertinent prior art, or considered material to the patentability of any claim of the present application.

### SUMMARY OF THE INVENTION

The present invention provides for the first time a highly effective therapeutic agent for the treatment of B-cell malignancies, including multiple myeloma. Multiple myeloma is incurable and conventional therapy results in complete remission in only 5% of patients with overall median survival only about 36 months. It is now disclosed that a human recombinant antibody specific to a dimeric FGFR3 extracellular domain is highly effective in preventing, attenuating or treating certain subtypes of multiple myeloma.

In one aspect the present invention relates to a method for the prevention, attenuation or treatment of multiple myeloma comprising administering a therapeutically effective amount of a molecule comprising the antigen-binding portion of an isolated antibody having specificity and affinity for FGFR3, the molecule inducing apoptosis of a myeloma cell, the myeloma cell expressing FGFR3 and a pharmaceutically acceptable carrier to a subject in need thereof.

Another aspect relates to the use of a molecule comprising the antigen-binding portion of an isolated antibody having specificity and affinity for FGFR3, the molecule inducing apoptosis of a myeloma cell, the myeloma cell expressing FGFR3, for the manufacture of a medicament for the treatment of multiple myeloma.

Another aspect of the present invention relates to a pharmaceutical composition for the prevention, attenuation or treatment of a B-cell malignancy comprising as an active ingredient a therapeutically effective amount of a molecule comprising the antigen-binding portion of an isolated antibody having specificity and affinity for FGFR3.

Some of the molecules and compositions thereof described herein have been disclosed in International patent application WO 02/102972, the teachings of which are incorporated by reference as if fully set forth herein, co-assigned to some of the applicants of the present invention. These compositions were disclosed previously as being useful for treating skeletal dysplasias and proliferative diseases. The molecules were shown to be effective in inhibiting both the wild type and constitutively activated forms of FGFR3. WO 02/102972 is a disclosure that does not anticipate the present claims for specific compounds useful for treating a specific indication. It is now disclosed that certain of said known compositions are especially effective in treating and attenuating multiple myeloma.

According to one embodiment of the present invention the molecule that comprises the antigen-binding portion of an antibody having specificity and affinity for fibroblast growth factor receptor 3 (FGFR3), is selected from a polyclonal antibody, a monoclonal antibody, a chimeric antibody, a single domain antibody, a recombinant antibody and fragments thereof. A preferred antibody species is a recombinant antibody. A more preferred antibody species is selected from a recombinant single chain antibody and a recombinant Fab antibody. Single chain antibodies can be single chain composite polypeptides having antigen binding capabilities and comprising amino acid sequences homologous or analogous to the variable regions of an immunoglobulin light and heavy chain i.e. linked V_{H}-V_{L} or single chain Fv (scFv).

In certain embodiments the present invention provides a method of preventing, attenuating or treating multiple myeloma comprising administering a pharmaceutical composition comprising a molecule comprising the antigen-binding portion of an antibody having specificity and affinity for fibroblast growth factor receptor 3 (FGFR3), the molecule comprising a V_{H}-CDR3 region having a polypeptide sequence as set forth in anyone of SEQ ID NOS: 1-9 and a V_{L}-CDR3 region having a polypeptide sequence as set forth in anyone of SEQ ID NOS: 10-18, and a pharmaceutically acceptable carrier. The corresponding polynucleotide sequences of the V_{H}-CDR3 and V_{L}-CDR3 regions are set forth in SEQ ID NOS: 39-47 and SEQ ID NOS: 48-56, respectively. These sequences have been disclosed in WO 02/102972, assigned to some of the assignees of the present invention.

According to one preferred embodiment the molecule comprising the antigen-binding portion of an antibody having specificity and affinity for fibroblast growth factor receptor 3 (FGFR3) comprises a V_{H}-CDR3 region having a polypeptide sequence as set forth in SEQ ID NO: 1 and a V_{L}-CDR3 region having a polypeptide sequence as set forth in SEQ ID NO: 10, and a pharmaceutically acceptable carrier. The corresponding polynucleotide sequences of the V_{H}-CDR3 and V_{L}-CDR3 regions are set forth in SEQ ID NO: 39 and SEQ ID NO: 48, respectively.

Another preferred embodiment of the present invention is a pharmaceutical composition for the prevention, attenuation or treatment of multiple myeloma comprising the antigen-binding portion of an antibody having specificity and affinity for fibroblast growth factor receptor 3 (FGFR3) comprising a V_{H}-CDR3 region having a polypeptide sequence as set forth in SEQ ID NO: 1 and a V_{L}-CDR3 region having a polypeptide sequence as set forth in SEQ ID NO: 10, and a pharmaceutically acceptable carrier (designated as PRO-001).

According to various additional embodiments the present invention provides a method of preventing, attenuating or treating a multiple myeloma comprising administering a composition comprising a therapeutically effective molecule comprising the antigen-binding portion of an antibody having specificity and affinity for fibroblast growth factor receptor 3, the molecule comprising a V_{H} domain having a polypeptide sequence as set forth in anyone of SEQ ID NOS: 19-27 and the V_{L} domains having a polypeptide sequence as set forth in anyone of SEQ ID NOS: 28-36, and a pharmaceutically acceptable carrier. The corresponding polynucleotide sequences of the V_{H} and V_{L} domains are set forth in SEQ ID NOS: 57-65 and SEQ ID NOS: 66-74, respectively.

According to certain preferred embodiments the molecule comprising the antigen-binding portion of an antibody having specificity and affinity for fibroblast growth factor receptor 3 comprises a V_{H} domain having a polypeptide sequence as set forth in SEQ ID NO: 19 and the V_{L} domain having a polypeptide sequence as set forth in SEQ ID NO: 28, and a pharmaceutically acceptable carrier. The corresponding polynucleotide sequences of the V_{H} and V_{L} domains are set forth in SEQ ID NO: 57 and SEQ ID NO: 66, respectively.

In yet another preferred embodiment the pharmaceutical composition comprises a single chain Fv molecule (scFv) having a polypeptide sequence set forth in SEQ ID NO: 37 having corresponding polynucleotide sequence SEQ ID NO: 38, and a pharmaceutically acceptable carrier.

The present invention also provides pharmaceutical compositions comprising one or more PEGylated antibodies and fragments thereof which immunospecifically bind to FGFR3. Wherein the PEGylated antibodies and fragments thereof retain the biological activity of the native molecules as determined by their ability to bind and neutralize FGFR3.

In one embodiment, a pharmaceutical composition of the invention comprises a PEGylated single chain Fv molecule (scFv) having a polypeptide sequence set forth in SEQ ID NO: 37 wherein leucine, the original amino acid at the N-terminus is replaced with serine to allow targeted PEGylation.

In certain embodiments the affinity of the molecule comprising an antigen binding domain of an antibody is measured by methods known in the art including binding assays and BIAcore (biomolecular interaction analyzing system). According to certain embodiments, affinity of the antigen binding domain of an antibody is less than about 30 nM as measured in a BIAcore reactor, preferably less than about 15 nm and more preferably less than about 5 nm.

In another embodiment the pharmaceutical composition of the present invention is administered to the patient in combination with another therapeutic agent. Such other therapeutic agent may be an antibody or a chemotherapeutic agent. Chemotherapeutic agents are commonly used in the treatment of multiple myeloma and may include (but are not limited to) melphalan, doxorubicin, carmustine, cyclophosphamide, thalidomide, bortezomib and lenalidomide.

### BRIEF DESCRIPTION OF THE FIGURES

FIG. 1A is a flow cytometry chart showing B9-FGFR WT (wild type) cells fluorescently labeled with PRO 001 followed by a PE-conjugated anti-human secondary antibody. The filled histogram indicates parental B9 cells (lacking FGFR3 expression); the dotted light line, B9-FGFR WT without aFGF; the solid dark line, B9-WT in the presence of aFGF. The Y axis represents counts indicating the amount of cells. The X axis represents fluorescence intensity.

FIG. 1B is a graph showing viability of B9-FGFR WT cells treated with different concentrations of PRO-001. The filled bars represent the control (no PRO-001). The dotted bars represent PRO-001 treated cells.

FIG. 1C is a photograph of a Western blot showing immune staining of RCJ-FGFR3 cell lysates. RCJ cells were stimulated with FGF (+) with or without pre-incubation with a Fab. Lane 1—no FGF stimulation and no pre-incubation with a Fab. Lane 2—FGF stimulation, no pre-incubation with a Fab. Lane 3—FGF stimulation and pre incubation with a control (C) antibody. Lane 4—FGF stimulation and pre-incubation with an anti-FGFR3 (001) Fab. The mid panel (Phospho-JNK) shows total cell lysates probed with anti-Phospho-JNK antibodies. The upper panel (Phospho-FGFR3) shows cell lysates immunoprecipitated (IP) with anti-FGFR3 antibody and then analyzed by Western blot with anti-phosphotyrosine (4G10). The lower panel (FGFR3) shows cell lysates immunoprecipitated (IP) with anti-FGFR3 antibody and then analyzed by Western blot with anti-FGFR3.

FIG. 1D is a graph showing proliferation of FGFR expressing FDCP cells in the presence of increasing concentrations of PRO-001 as determined by XTT analysis. Data are the average of duplicate cultures. The X axis represents concentration of PRO-001 Fab. The Y axis represents % inhibition.

FIG. 2 is a graph showing the viability of human myeloma cell lines in the presence of PRO-001. Viability is reported as the ratio between the optical density (OD) in the presence of FGF±inhibitor and the OD in the absence of FGF.

FIG. 3 is a flow cytometry chart showing phosphorylation of ERK (Extracellular signal-regulated protein kinase) in UTMC2 cells. The filled histogram represents UTMC2 without FGF stimulation (unstimulated); the light line represents cells stimulated with FGF and treated with a control antibody (FGF/vehicle); the dark line represents cells stimulated with FGF and treated with PRO-001 (FGF/PRO-001).

FIG. 4 is a graph showing viability of UTMC2 cells treated with FGF, IL6 or IGF-1. The dark bars represent stimulated cells treated with a control antibody; the dotted bar represents stimulated cells treated with 5 μg/ml PRO-001; the square-filled bar represents stimulated cells treated with 100 nM PD173074.

FIG. 5 is a graph showing apoptosis of UTMC2 cells in response to treatment with PRO-001 in the presence of BMSCs (stroma). BMSCs alone (stroma) or BMSCs together with UTMC2 cells (stroma/UTMC2) were cultured with control antibody or 5 μg/ml PRO-001 for 72 hours and apoptosis was assessed by means of a flow cytometry assay of annexin V binding and propidium iodide exclusion. Values represent means of quadruplicate cultures ±SD.

FIGS. 6 A-B are flow cytometry charts of human primary myeloma cells treated with anti-FGFR antibody PRO-001. A: Freshly isolated BMSCs were stained with PRO-001 (black line) or control antibody (grey line) and then stained with PE-conjugated anti-human secondary antibody. B: Primary myeloma cells were incubated in the absence (filled) or presence of FGF (light line) or pre-incubated with 5 μg/ml PRO-001 (dark line) for 2 h and then stimulated with FGF. ERK1/2 phosphorylation was assessed by flow cytometry analysis.

FIG. 7 is a flow cytometry chart showing CD138 positive primary MM cells stained with Annexin V. Primary myeloma cells were cultured in the presence of control Fab (lower panel) or 5 μg/ml PRO-001 (upper panel). Cells were harvested after 7 days, stained with annexin V-FITC and analyzed by flow cytometry. Myeloma cells were identified as CD138⁺⁺. The total percentage of CD138⁺⁺ cells is shown in the upper left quadrant. Shown is a representative experiment.

FIG. 8A is a graph showing viability of FDCP-FGFR3^{S249C}. Cells were cultured in the presence of increasing amounts of PRO-001 or a control antibody (C) for two days. Cell proliferation was determined by XTT analysis. Data are the average of duplicate cultures.

FIG. 8B is a graph showing the effect of PRO-001 on an FGFR3-driven xenograft tumor model. Nude mice (3 in each group), were injected S.C. at 2 locations, one on each flank (a—right flank, b—left flank), with 2×10⁶ FDCP-FGFR3^{S249C} cells each. A week later, mice were randomized to receive PRO-001 by I.P. injection according to the schedule described in Table I or PBS as control. Tumor volume was estimated from measurements in 3 dimensions at 22 or 29 days post cell injection.

FIG. 9 is a graph showing FGFR3 binding activity of PRO-001Ser scFv. A MaxiSorp plate was coated with the indicated amount of single chain. Soluble FGFR3/Fc was added and bound receptor was measured with HRP-anti-Fc.

FIG. 10 is a photograph of a coomassie stained SDS-PAGE showing specific FGFR3 binding of mPEG-HZ5K, mPEG-HZ20K and mPEG-HZ40K conjugated PRO-001Ser. The PEGylation reaction mix (P) was incubated with FGFR3/Fc or FGFR1/Fc-protein A-sepharose beads. The unbound material was collected and incubated consecutively 2 more times with fresh beads. The bound fractions (B1, B2 and B3) as well as the unbound material (U2) from the last binding cycle were analyzed by coomassie stained SDS-PAGE. U—unmodified single chain.

FIG. 11 is a graph showing FGFR3 neutralizing activity of PRO-001-PEG conjugates. PRO-001Ser PEGylated with mPEG-HZ-5K, mPEG-HZ-20K or mPEG-HZ-40K were analyzed by XTT using FDCP-FGFR3 cells or FDCP-FGFR1 cells as control.

### DETAILED DESCRIPTION OF THE INVENTION

The present invention is based on the discovery that an antibody having specificity and affinity for fibroblast growth factor receptor 3 (FGFR3) induces apoptosis of myeloma cells, in vitro and in vivo.

The present invention relates to a method of treating a B-cell malignancy comprising administering a pharmaceutical composition comprising a therapeutically effective amount of a molecule comprising the antigen-binding portion of an isolated antibody having specificity and affinity for FGFR3, the molecule inducing apoptosis of a myeloma cell, and a pharmaceutically acceptable carrier to a subject in need thereof. In one preferred embodiment the B-cell malignancy is multiple myeloma.

The present invention further relates to the use of at least one anti-FGFR3 antibody for the manufacture of a medicament for the prevention, attenuation or treatment of a B-cell malignancy, preferably multiple myeloma.

Without wishing to be bound to any particular theory, the anti-FGFR3 antibody may interfere with adhesion between the stroma and the myeloma cell. This interaction is crucial for growth of the myeloma plasma cells and disease progression and results in local manifestations such as lytic bone disease and systemic manifestations such as immunocompromise and anemia.

International patent application WO 02/102972, co-assigned to some of the assignees of the present invention, discloses monoclonal antibodies to receptor protein tyrosine kinases, including specific anti-Fibroblast Growth Factor Receptor 3 (FGFR3) antibodies. Utilizing a soluble dimeric form of the extracellular domain of the FGFR3 receptor to screen for antibodies (e.g., Fabs) from a phage display antibody library yielded numerous high affinity (K_{D}<50 nM) antibodies (Fabs) that bind FGFR3 and interfere with ligand binding, thereby blocking ligand-dependent activation of FGFR3. Certain antibodies were shown to be specific for FGFR3 and useful to neutralize FGFR3 activity and for the treatment of skeletal dysplasias such as achondroplasia and proliferative diseases such as bladder cancer. Additional antibodies useful for blocking ligand-independent, or constitutive, activation were also identified and isolated.

The present inventors have now discovered that certain molecules disclosed in that application are highly effective in inducing apoptosis in FGFR3 expressing myeloma cells, in particular myeloma cells situated in the bone marrow stroma of a multiple myeloma patient. These molecules are now disclosed for the prevention, attenuation and treatment of multiple myeloma.

For convenience certain terms employed in the specification, examples and claims are described herein.

The term “fibroblast growth factor receptor” or “FGFR” denotes a receptor specific for FGF which is necessary for transducing the signal exerted by FGF to the cell interior, typically comprising an extracellular ligand-binding domain, a single transmembrane helix, and a cytoplasmic domain having tyrosine kinase activity. The FGFR extracellular domain consists of three immunoglobulin-like (Ig-like) domains (D1, D2 and D3), a heparin binding domain and an acidic box. Four FGFR genes that encode for multiple receptor protein variants are known. Alternative splicing of the FGFR3 mRNAs generates at least two known isoforms of the receptors, FGFR3IIIc and FGFR3IIIb.

Throughout the specification and the claims that follow, the term “FGFR3 specific” refers to any effector that has higher affinity or activity or binding to FGFR3 polypeptide or to the polynucleotide encoding same, than to another FGF receptor protein or polynucleotide. The effector can be any molecule including a ligand, an inhibitor, an antibody, a polypeptide, a polynucleotide or a small organic molecule such as a tyrosine kinase inhibitor. It is to be explicitly understood that the term “FGFR3 specific” does not exclude or preclude situations wherein the effector has some activity on another FGF receptor subtype. It is further to be understood that if the activity mediated via another receptor subtype is clinically important for the therapeutic utility observed, this is explicitly encompassed within the scope of the claimed invention.

As used herein, “affinity” refers to the strength of the reaction of a single antigen-combining site with a monovalent antigenic determinant. Affinity is measured as the binding constant.

Specificity of an antibody is the property of an antibody which enables it to react with some antigenic determinants and not with others. Specificity is dependent on chemical composition, physical forces, and molecular structure at the binding site.

As used herein “multiple myeloma” also known as plasma cell myeloma refers to the proliferative hematologic disease of the plasma cell. Multiple myeloma is characterized by excessive numbers of abnormal plasma cells in the bone marrow and overproduction of intact monoclonal immunoglobulin (IgG, IgA, IgD, or IgE) or Bence-Jones protein. Hypercalcemia, anemia, renal damage, increased susceptibility to bacterial infection, and impaired production of normal immunoglobulin are common clinical manifestations of multiple myeloma. It is often also characterized by diffuse osteoporosis and lytic bone lesions predominantly of the axial skeleton.

As used herein “stroma” refers to the cells and the supporting tissue around the myeloma cells in the bone marrow. Adhesion of the myeloma cells to the bone marrow enhances the growth of myeloma.

One aspect of the present invention is directed to a method of preventing, attenuating or treating multiple myeloma by administering a molecule comprising the antigen-binding portion of an antibody which diminishes or inhibits activation of FGFR3, and a pharmaceutically acceptable carrier. According to one embodiment of the present invention the antigen-binding portion of an antibody is directed to the extracellular domain of the FGFR3.

One embodiment of the present invention is directed to molecules comprising an antigen binding domain which blocks ligand-dependent activation of FGFR3.

The molecule having the antigen-binding portion of an antibody according to the present invention is useful for blocking the ligand-dependent activation and/or ligand independent (constitutive) activation of FGFR3. Preferred embodiments of such antibodies/molecules, obtained from an antibody library designated as HuCAL® (Human Combinatorial Antibody Library) clone, are presented in Table 1 with the unique V_{H}-CDR3 and V_{L}-CDR3 sequences presented in Table 2.

TABLE 1
Properties of antibodies useful for inhibiting,
treating or attenuating multiple myeloma.

| | Affinity to | Affinity to | | | IC₅₀ | |
|---|---|---|---|---|---|---|
| | FGFR3 | FGFR3 | Affinity to | | FGFR3 | FGFR3 |
| | (BIAcore) | (FACS) | FGFR1 | K_{off} | (FGF9) | Domain |
| Clone | nM | nM | nM | (s⁻¹) | nM | Specificity |
| PRO-001 | 1.5 | 0.7 | — | 7.1 × 10e−4 | 19 | 2 |
| PRO-002 | 37 | 43 | — | 2 × 10e−2 | 360 | 2 |
| PRO-012 | 14 | 6.5 | — | 2.3 × 10e−3 | 58 | 2 |
| PRO-021 | 9 | 1.1 | — | 3.6 × 10e−3 | 50 | 3c |
| PRO-024 | 10 | NA | — | 5.4 × 10e−3 | 70 | 3c |
| PRO-026 | 4 | 1.4 | 32 | 5 × 10e−4 | 70 | 3c |
| PRO-029 | 6 | <1 | 29 | 1.4 × 10e−3 | 20 | 3c |
| PRO-054 | 3.7 | NA | 2.5 | 2 × 10e−3 | 45 | 3c |
| PRO-055 | 2.9 | NA | — | 7.4 × 10e−4 | 34 | 3c |

Key: affinity (nM) of the respective molecules to FGFR3 and FGFR1 was measured by BIAcore and/or FACS. IC₅₀ were determined for the dimeric dHLX format of certain molecule with antigen binding site in an FDCP-FGFR3 proliferation assay performed with FGF9. Fab-dHLX refers to a Fab mini-antibody format where a dimer of the Fab monomer is produced as a fusion protein after insertion into an expression vector. The values obtained by BIAcore demonstrated that the interactions between antibody and receptor are specific.

TABLE 2

V_{H}-CDR3 and corresponding V_{L}-CDR3 polypeptide
sequences

| | | |
|---|---|---|
| Clone | V_{H}-CDR3 | V_{L}-CDR3 |

| | | |
|---|---|---|
| PRO-001 | SYYPDFDY | QSYDGPDLW |
| | (SEQ ID NO:1) | (SEQ ID NO:10) |
| PRO-002 | DFLGYEFDY | QSYDYSADY |
| | (SEQ ID NO:2) | (SEQ ID NO:11) |
| PRO-012 | YHSWYEMGYY GSTVGYMFDY | QSYDFDFA |
| | (SEQ ID NO:3) | (SEQ ID NO:12) |
| PRO-021 | DNWFKPFSDV | QQYDSIPY |
| | (SEQ ID NO:4) | (SEQ ID NO:13) |
| PRO-024 | VNHWTYTFDY | QQMSNYPD |
| | (SEQ ID NO:5) | (SEQ ID NO:14) |
| PRO-026 | GYWYAYFTYI NYGYFDN | QSYDNNSDV |
| | (SEQ ID NO:6) | (SEQ ID NO:15) |
| PRO-029 | TWQYSYFYYL DGGYYFDI | QQTNNAPV |
| | (SEQ ID NO:7) | (SEQ ID NO:16) |
| PRO-054 | NMAYTNYQYV NNPHFDY | QSYDYFKL |
| | (SEQ ID NO:8) | (SEQ ID NO:17) |
| PRO-055 | SMNSTNYWYL RRVLFDH | QSYDMYMYI |
| | (SEQ ID NO:9) | (SEQ ID NO:18) |

V_{H} refers to the variable heavy chain, V_{L} refers to the variable light chain, CDR3 refers to complementarity determining region 3. In certain preferred embodiments the present invention provides a method of treating or preventing multiple myeloma comprising administering a composition comprising a therapeutically effective molecule c Comprising a V_{H}-CDR3 region having a polypeptide sequence as set forth in any one of SEQ ID NOS: 1-9 and a corresponding V_{L}-CDR3 region having a polypeptide sequence as set forth in any one of SEQ ID NOS: 10-18, and a pharmaceutically acceptable carrier. The corresponding polynucleotide sequences of the V_{H}-CDR3 and V_{L}-CDR3 regions as set forth in any one of SEQ ID NOS: 39-47 and SEQ ID NOS: 48-56, respectively. The polynucleotide sequences are presented in Table 3.

According to certain embodiments the present invention provides a method of treating or preventing multiple myeloma comprising administering a composition comprising a therapeutically effective molecule comprising a V_{H} domain having a polypeptide sequence as set forth in any one of SEQ ID NOS: 19-27 and the corresponding V_{L} domains having a polypeptide sequence as set forth in any one of SEQ ID NOS: 28-36, and a pharmaceutically acceptable carrier. The preferred V_{H} and V_{L} sequences are presented herein.

| PRO-001-VH | |
|---|---|
| (SEQ ID NO: 19) | |
| 1 | QVQLQQSGPG LVKPSQTLSL TCAISGDSVS SNSAAWNWIR QSPGRGLEWL |
| 51 | GRTYYRSKWY NDYAVSVKSR ITINPDTSKN QFSLQLNSVT PEDTAVYYCA |
| 101 | RSYYPDFDYW GQGTLVTVSS |
| PRO-002-VH | |
| (SEQ ID NO: 20) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARDF |
| 101 | LGYEFDYWGQ GTLVTVSS |
| PRO-012-VH | |
| (SEQ ID NO: 21) | |
| 1 | QVQLKESGPA LVKPTQTLTL TCTFSGFSLS TSGVGVGWIR QPPGKALEWL |
| 51 | ALIDWDDDKY YSTSLKTRLT ISKDTSKNQV VLTMTNMDPV DTATYYCARY |
| 101 | HSWYEMGYYG STVGYMFDYW GQGTLVTVSS |
| PRO-021-VH | |
| (SEQ ID NO: 22) | |
| 1 | QVQLVQSGAE VKKPGSSVKV SCKASGGTFS SYAISWVRQA PGQGLEWMGG |
| 51 | IIPIFGTANY AQKFQGRVTI TADESTSTAY MELSSLRSED TAVYYCARDN |
| 101 | WFKPFSDVWG QGTLVTVSS |
| PRO-024-VH | |
| (SEQ ID NO: 23) | |
| 1 | QVQLVQSGAE VKKPGSSVKV SCKASGGTFS SYAISWVRQA PGQGLEWMGG |
| 51 | IIPIFGTANY AQKFQGRVTI TADESTSTAY MELSSLRSED TAVYYCARVN |
| 101 | HWTYTFDYWG QGTLVTVSS |
| PRO-026-VH | |
| (SEQ ID NO: 24) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARGY |
| 101 | WYAYFTYINY GYFDNWGQGT LVTVSS |
| PRO-029-VH | |
| (SEQ ID NO: 25) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARTW |
| 101 | QYSYFYYLDG GYYFDIWGQG TLVTVSS |
| PRO-054-VH | |
| (SEQ ID NO: 26) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARNM |
| 101 | AYTNYQYVNM HFDYWGQGT LVTVSS |
| PRO-055-VH | |
| (SEQ ID NO: 27) | |
| 1 | QVQLVQSGAE VKKPGASVKV SCKASGYTFT SYYMHWVRQA PGQGLEWMGW |
| 51 | INPNSGGTNY AQKFQGRVTM TRDTSISTAY MELSSLRSED TAVYYCARSM |
| 101 | NSTMYWYLRR VLFDGWGQGT LVTVSS |
| PRO-001-VL | |
| (SEQ ID NO: 28) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DGPDLWVFGG |
| 101 | GTKLTVLGQ |
| PRO-002-VL | |
| (SEQ ID NO: 29) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DYSADYVFGG |
| 101 | GTKLTVLGQ |
| PRO-012-VL | |
| (SEQ ID NO: 30) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DFDFAVFGGG |
| 101 | TKLTVLGQ |
| PRO-021-VL | |
| (SEQ ID NO: 31) | |
| 1 | DIVMTQSPDS LAVSLGERAT INCRSSQSVL YSSNNKNYLA WYQQKPGQPP |
| 51 | KLLIYWASTR ESGVPDRFSG SGSGTDFTLT ISSLQAEDVA VYYCQQYDSI |
| 101 | PYTFGQGTKV EIKRT |
| PRO-024-VL | |
| (SEQ ID NO: 32) | |
| 1 | DIVLTQSPAT LSLSPGERAT LSCRASQSVS SSYLAWYQQK PGQAPRLLIY |
| 51 | GASSRATGVP ARFSGSGSGT DFTLTISSLE PEDFATYYCQ QMSNYPDTFG |
| 101 | QGTKVEIKRT |
| MS-Pro-26-VL | |
| (SEQ ID NO: 33) | |
| 1 | DIALTQPASV SGSPGQSITI SCTGTSSDVG GYNYVSWYQQ HPGKAPKLMI |
| 51 | YDVSNRPSGV SNRFSGSKSG NTASLTISGL QAEDEADYYC QSYDNNSDVV |
| 101 | FGGGTKLTVL GQ |
| PRO-029-VL | |
| (SEQ ID NO: 34) | |
| 1 | DIVLTQSPAT LSLSPGERAT LSCRASQSVS SSYLAWYQQK PGQAPRLLIY |
| 51 | GASSRATGVP ARFSGSGSGT DFTLTISSLE PEDFATYYCQ QTNNAPVTFG |
| 101 | QGTKVEIKRT |
| PRO-054-VL | |
| (SEQ ID NO: 35) | |
| 1 | DIELTQPPSV SVAPGQTARI SCSGDALGDK YASWYQQKPG QAPVLVIYDD |
| 51 | SDRPSGIPER FSGSNSGNTA TLTISGTQAE DEADYYCQSY DYFKLVFGGG |
| 101 | TKLTVLGQ |
| PRO-055-VL | |
| (SEQ ID NO: 36) | |
| 1 | DIALTQPASV SGSPGQSITI SCTGTSSDVG GYNYVSWYQQ HPGKAPKLMI |
| 51 | YDVSNRPSGV SNRFSGSKSG NTASLTISGL QAEDEADYYC QSYDMYNYIV |
| 101 | FGGGTKLTVL GQ |

The corresponding polynucleotide sequences of the V_{H} and V_{L} domains have SEQ ID NOS: 57-65 and SEQ ID NOS: 66-74, respectively.

<SEQ ID NO: 57; DNA> PRO-001 VH
CAGGTGCAATTGCAACAGTCTGGTCCGGGCCTGGTGAAACCGAGCCAAAC
CCTGAGCCTGACCTGTGCGATTTCCGGAGATAGCGTGAGCAGCAACAGCG
CGGCGTGGAACTGGATTCGCCAGTCTCCTGGGCGTGGCCTCGAGTGGCTG
GGCCGTACCTATTATCGTAGCAAATGGTATAACGATTATGCGGTGAGCGT
GAAAAGCCGGATTACCATCAACCCGGATACTTCGAAAAACCAGTTTAGCC
TGCAACTGAACAGCGTGACCCCGGAAGATACGGCCGTGTATTATTGCGCG
CGTTCTTATTATCCTGATTTTGATTATTGGGGCCAAGGCACCCTGGTGAC
GGTTAGCTCAGC
<SEQ ID NO: 58; DNA> PRO-002 VH
CAGGTGCAATTGGTTCAGAGCGGCGCGGAAGTGAAAAAACCGGGCGCGAG
CGTGAAAGTGAGCTGCAAAGCCTCCGGATATACCTTTACCAGCTATTATA
TGCACTGGGTCCGCCAAGCCCCTGGGCAGGGTCTCGAGTGGATGGGCTGG
ATTAACCCGAATAGCGGCGGCACGAACTACGCGCAGAAGTTTCAGGGCCG
GGTGACCATGACCCGTGATACCAGCATTAGCACCGCGTATATGGAACTGA
GCAGCCTGCGTAGCGAAGATACGGCCGTGTATTATTGCGCGCGTGATTTT
CTTGGTTATGAGTTTGATTATTGGGGCCAAGGCACCCTGGTGACGGTTAG
CTCAGC
<SEQ ID NO: 59; DNA> PRO-012 VH
CAGGTGCAATTGAAAGAAAGCGGCCCGGCCCTGGTGAAACCGACCCAAAC
CCTGACCCTGACCTGTACCTTTTCCGGATTTAGCCTGTCCACGTCTGGCG
TTGGCGTGGGCTGGATTCGCCAGCCGCCTGGGAAAGCCCTCGAGTGGCTG
GCTCTGATTGATTGGGATGATGATAAGTATTATAGCACCAGCCTGAAAAC
GCGTCTGACCATTAGCAAAGATACTTCGAAAAATCAGGTGGTGCTGACTA
TGACCAACATGGACCCGGTGGATACGGCCACCTATTATTGCGCGCGTTAT
CATTCTTGGTATGAGATGGGTTATTATGGTTCTACTGTTGGTTATATGTT
TGATTATTGGGGCCAAGGCACCCTGGTGACGGTTAGCTCAGC
<SEQ ID NO: 60; DNA> PRO-021 VH
CAGGTGCAATTGGTTCAGTCTGGCGCGGAAGTGAAAAAACCGGGCAGCAG
CGTGAAAGTGAGCTGCAAAGCCTCCGGAGGCACTTTTAGCAGCTATGCGA
TTAGCTGGGTGCGCCAAGCCCCTGGGCAGGGTCTCGAGTGGATGGGCGGC
ATTATTCCGATTTTTGGCACGGCGAACTACGCGCAGAAGTTTCAGGGCCG
GGTGACCATTACCGCGGATGAAAGCACCAGCACCGCGTATATGGAACTGA
GCAGCCTGCGTAGCGAAGATACGGCCGTGTATTATTGCGCGCGTGATAAT
TGGTTTAAGCCTTTTTCTGATGTTTGGGGCCAAGGCACCCTGGTGACGGT
TAGCTCAGC
<SEQ ID NO: 61; DNA> PRO-024 VH
CAGGTGCAATTGGTTCAGTCTGGCGCGGAAGTGAAAAAACCGGGCAGCAG
CGTGAAAGTGAGCTGCAAAGCCTCCGGAGGCACTTTTAGCAGCTATGCGA
TTAGCTGGGTGCGCCAAGCCCCTGGGCAGGGTCTCGAGTGGATGGGCGGC
ATTATTCCGATTTTTGGCACGGCGAACTACGCGCAGAAGTTTCAGGGCCG
GGTGACCATTACCGCGGATGAAAGCACCAGCACCGCGTATATGGAACTGA
GCAGCCTGCGTAGCGAAGATACGGCCGTGTATTATTGCGCGCGTGTTAAT
CATTGGACTTATACTTTTGATTATTGGGGCCAAGGCACCCTGGTGACGGT
TAGCTCAGC
<SEQ ID NO: 62; DNA> PRO-026 VH
CAGGTGCAATTGGTTCAGAGCGGCGCGGAAGTGAAAAAACCGGGCGCGAG
CGTGAAAGTGAGCTGCAAAGCCTCCGGATATACCTTTACCAGCTATTATA
TGCACTGGGTCCGCCAAGCCCCTGGGCAGGGTCTCGAGTGGATGGGCTGG
ATTAACCCGAATAGCGGCGGCACGAACTACGCGCAGAAGTTTCAGGGCCG
GGTGACCATGACCCGTGATACCAGCATTAGCACCGCGTATATGGAACTGA
GCAGCCTGCGTAGCGAAGATACGGCCGTGTATTATTGCGCGCGTGGTTAT
TGGTATGCTTATTTTACTTATATTAATTATGGTTATTTTGATAATTGGGG
CCAACGCACCCTGGTGACGGTTAGCTCAGC
<SEQ ID NO: 63; DNA> PRO-029 VH
CAGGTGCAATTGGTTCAGAGCGGCGCGGAAGTGAAAAAACCGGGCGCGAG
CGTGAAAGTGAGCTGCAAAGCCTCCGGATATACCTTTACCAGCTATTATA
TGCACTGGGTCCGCCAAGCCCCTGGGCAGGGTCTCGAGTGGATGGGCTGG
ATTAACCCGAATAGCGGCGGCACGAACTACGCGCAGAAGTTTCAGGGCCG
GGTGACCATGACCCGTGATACCAGCATTAGCACCGCGTATATGGAACTGA
GCAGCCTGCGTAGCGAAGATACGGCCGTGTATTATTGCGCGCGTACTTGG
CAGTATTCTTATTTTTATTATCTTGATGGTGGTTATTATTTTGATATTTG
GGGCCAAGGCACCCTGGTGACGGTTAGCTCAGC
<SEQ ID NO: 64; DNA> PRO-054 VH
CAGGTGCAATTGGTTCAGAGCGGCGCGGAAGTGAAAAAACCGGGCGCGAG
CGTGAAAGTGAGCTGCAAAGCCTCCGGATATACCTTTACCAGCTATTATA
TGCACTGGGTCCGCCAAGCCCCTGGGCAGGGTCTCGAGTGGATGGGCTGG
ATTAACCCGAATAGCGGCGGCACGAACTACGCGCAGAAGTTTCAGGGCCG
GGTGACCATGACCCGTGATACCAGCATTAGCACCGCGTATATGGAACTGA
GCAGCCTGCGTAGCGAAGATACGGCCGTGTATTATTGCGCGCGTAATATG
GCTTATACTAATTATCAGTATGTTAATATGCCTCATTTTGATTATTGGGG
CCAAGGCACCCTGGTGACGGTTAGCTCAGC
<SEQ ID NO: 65; DNA> PRO-055 VH
CAGGTGCAATTGGTTCAGAGCGGCGCGGAAGTGAAAAAACCGGGCGCGAG
CGTGAAAGTGAGCTGCAAAGCCTCCGGATATACCTTTACCAGCTATTATA
TGCACTGGGTCCGCCAAGCCCCTGGGCAGGGTCTCGAGTGGATGGGCTGG
ATTAACCCGAATAGCGGCGGCACGAACTACGCGCAGAAGTTTCAGGGCCG
GGTGACCATGACCCGTGATACCAGCATTAGCACCGCGTATATGGAACTGA
GCAGCCTGCGTAGCGAAGATACGGCCGTGTATTATTGCGCGCGTTCTATG
AATTCTACTATGTATTGGTATCTTCGTCGTGTTCTTTTTGATCATTGGGG
CCAAGGCACCCTGGTGACGGTTAGCTCAGC
<SEQ ID NO: 66> DNA> PRO-001 VL
GATATCGAACTGACCCAGCCGCCTTCAGTGAGCGTTGCACCAGGTCAGAC
CGCGCGTATCTCGTGTAGCGGCGATGCGCTGGGCGATAAATACGCGAGCT
GGTACCAGCAGAAACCCGGGCAGGCGCCAGTTCTGGTGATTTATGATGAT
TCTGACCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTGCCAGAGCTATGACGGTCCTGATCTTTGGGTGTTTGGCGGC
GGCACGAAGTTAACCGTTCTTGGCCAG
<SEQ ID NO: 67; DNA> PRO-002 VL
GATATCGAACTGACCCAGCCGCCTTCAGTGAGCGTTGCACCAGGTCAGAC
CGCGCGTATCTCGTGTAGCGGCGATGCGCTGGGCGATAAATACGCGAGCT
GGTACCAGCAGAAACCCGGGCAGGCGCCAGTTCTGGTGATTTATGATGAT
TCTGACCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTCCCAGAGCTATGACTATTCTGCTGATTATGTGTTTGGCGGC
GGCACGAAGTTAACCGTTCTTGGCCAG
<SEQ ID NO: 68; DNA> PRO-012 VL
GATATCGAACTGACCCAGCCGCCTTCAGTGAGCGTTGCACCAGGTCAGAC
CGCGCGTATCTCGTGTAGCGGCGATGCGCTGGGCGATAAATACGCGAGCT
GGTACCAGCAGAAACCCGGGCAGGCGCCAGTTCTGGTGATTTATGATGAT
TCTGACCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTGCCAGAGCTATGACTTTGATTTTGCTGTGTTTGGCGGCGGC
ACGAAGTTAACCGTTCTTGGCCAG
<SEQ ID NO: 69; DNA> PRO-021 VL
GATATCGTGATGACCCAGAGCCCGGATAGCCTGGCGGTGAGCCTGGGCGA
ACGTGCGACCATTAACTGCAGAAGCAGCCAGAGCGTGCTGTATAGCAGCA
ACAACAAAAACTATCTGGCGTGGTACCAGCAGAAACCAGGTCAGCCGCCG
AAACTATTAATTTATTGGGCATCCACCCGTGAAAGCGGGGTCCCGGATCG
TTTTAGCGGCTCTGGATCCGGCACTGATTTTACCCTGACCATTTCGTCCC
TGCAAGCTGAAGACGTGGCGGTGTATTATTGCCAGCAGTATGATTCTATT
CCTTATACCTTTGGCCAGGGTACGAAAGTTGAAATTAAACGTACG
<SEQ ID NO: 70; DNA> PRO-024 VL
GATATCGTGCTGACCCAGAGCCCGGCGACCCTGAGCCTGTCTCCGGGCGA
ACGTGCGACCCTGAGCTGCAGAGCGAGCCAGAGCGTGAGCAGCAGCTATC
TGGCGTGGTACCAGCAGAAACCAGGTCAAGCACCGCGTCTATTAATTTAT
GGCGCGAGCAGCCGTGCAACTGGGGTCCCGGCGCGTTTTAGCGGCTCTGG
ATCCGGCACGGATTTTACCCTGACCATTAGCAGCCTGGAACCTGAAGACT
TTGCGACTTATTATTGCCAGCAGATGTCTAATTATCCTGATACCTTTGGC
CAGGGTACGAAAGTTGAAATTAAACGTACG
<SEQ ID NO: 71; DNA> PRO-026 VL
GATATCGCACTGACCCAGCCAGCTTCAGTGAGCGGCTCACCAGGTCAGAG
CATTACCATCTCGTGTACGGGTACTAGCAGCGATGTGGGCGGCTATAACT
ATGTGAGCTGGTACCAGCAGCATCCCGGGAAGGCGCCGAAACTGATGATT
TATGATGTGAGCAACCGTCCCTCAGGCGTGAGCAACCGTTTTAGCGGATC
CAAAAGCGGCAACACCGCGAGCCTGACCATTAGCGGCCTGCAAGCGGAAG
ACGAAGCGGATTATTATTGCCAGAGCTATGACAATAATTCTGATGTTGTG
TTTGGCGGCGGCACGAAGTTAACCGTTCTTGGCCAG
<SEQ ID NO: 72; DNA> PRO-029 VL
GATATCGTGCTGACCCAGAGCCCGGCGACCCTGAGCCTGTCTCCGGGCGA
ACGTGCGACCCTGAGCTGCAGAGCGAGCCAGAGCGTGAGCAGCAGCTATC
TGGCGTGGTACCAGCAGAAACCAGGTCAAGCACCGCGTCTATTAATTTAT
GGCGCGAGCAGCCGTGCAACTGGGGTCCCGGCGCGTTTTAGCGGCTCTGG
ATCCGGCACGGATTTTACCCTGACCATTAGCAGCCTGGAACCTGAAGACT
TTGCGACTTATTATTGCCAGCAGACTAATAATGCTCCTGTTACCTTTGGC
CAGGGTACGAAAGTTGAAATTAAACGTACG
<SEQ ID NO: 73; DNA> PRO-054 VL
GATATCGAACTGACCCAGCCGCCTTCAGTGAGCGTTGCACCAGGTCAGAC
CGCGCGTATCTCGTGTAGCGGCGATGCGCTGGGCGATAAATACGCGAGCT
GGTACCAGCAGAAACCCGGGCAGGCGCCAGTTCTGGTGATTTATGATGAT
TCTGACCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTGCCAGAGCTATGACTATTTTAAGCTTGTGTTTGGCGGCGGC
ACGAAGTTAACCGTTCTTGGCCAG
<SEQ ID NO: 74; DNA> PRO-055 VL
GATATCGCACTGACCCAGCCAGCTTCAGTGAGCGGCTCACCAGGTCAGAG
CATTACCATCTCGTGTACGGGTACTAGCAGCGATGTGGGCGGCTATAACT
ATGTGAGCTGGTACCAGCAGCATCCCGGGAAGGCGCCGAAACTGATGATT
TATGATGTGAGCAACCGTCCCTCAGGCGTGAGCAACCGTTTTAGCGGATC
CAAAAGCGGCAACACCGCGAGCCTGACCATTAGCGGCCTGCAAGCGGAAG
ACGAAGCGGATTATTATTGCCAGAGCTATGACATGTATAATTATATTGTG
TTTGGCGGCGGCACGAAGTTAACCGTTCTTGGCCAG

In yet another preferred embodiment the pharmaceutical composition comprises a single chain Fv molecule (scFv) set forth in SEQ ID NO:37, having corresponding polynucleotide sequence SEQ ID NO:38, and a pharmaceutically acceptable carrier. The respective polypeptide and polynucleotide sequences are presented herein:

PRO-001 scFv polypeptide

(SEQ ID NO: 37)

MLTCAISGNS VSSNSAAWNW IRQSPGRGLE WLGRTYYRSK
WYNDYAVSVK SRITINPDTS KNQFSLQLNS VTPEDTAVYY
CARSYYPDFD YWGQGTLVTV SSAGGGSGGG GSGGGGSGGG
GSDIELTQPP SVSVAPGQTA RISCSGDALG DKYASWYQQK
PGQAPVLVIY DDSDRPSGIP ERFSGSNSGN TATLTISGTQ
AEDEADYYCQ SYDGPDLWVF GGGTKLTVLG QEFDYKMTMT
KRAVEPPAV
PRO-001 scFv DNA

(SEQ ID NO: 38)

1 ATGCTGACCT GTGCGATTTG CGGGAATAGC
GTGAGCAGCA ACAGCGCGGC GTGGAACTGG ATTCGCCAGT
CTCCTGGGCG TGGCCTCGAG TGGCTGGGCC GTACCTATTA
TCGTAGCAAA TGGTATAACG ATTATGCGGT GAGCGTGAAA
AGCCGGATTA CCATCAACCC GGATACTTCG AAAAACCAGT
TTAGCCTGCA ACTGAACAGC GTGACCCCGG AAGATACGGC
CGTGTATTAT TGCGCGCGTT CTTATTATCC TGATTTTGAT
TATTGGGGCC AAGGCACCCT GGTGACGGTT AGCTCAGCGG
GTGGCGGTTC TGGCGGCGGT GGGAGCGGTG GCGGTGGTTC
TGGCGGTGGT GGTTCCGATA TCGAACTGAC CCAGCCGCCT
TCAGTGAGCG TTGCACCAGG TCAGACCGCG CGTATCTCGT
GTAGCGGCGA TGCGCTGGGC GATAAATACG CGAGCTGGTA
CCAGCAGAAA CCCGGGCAGG CGCCAGTTCT GGTGATTTAT
GATGATTCTG ACCGTCCCTC AGGCATCCCG GAACGCTTTA
GCGGATCCAA CAGCGGCAAC ACCGCGACCC TGACCATTAG
CGGCACTCAG GCGGAAGACG AAGCGGATTA TTATTGCCAG
AGCTATGACG GTCCTGATCT TTGGGTGTTT GGCGGCGGCA
CGAAGTTAAC CGTTCTTGGC CAGGAATTCG ACTATAAGAT
GACGATGACA AAGCGCGCCG TGGAGCCACC CGCAGTTTGA

TABLE 3

V_{H}-CDR3 and corresponding V_{L}-CDR3 polynucleo-
tide sequence

| | | |
|---|---|---|
| Clone | V_{H}-CDR3 | V_{L}-CDR3 |

| | | |
|---|---|---|
| PRO- | TCTTATTATC CTGATTTTGA | CAGAGCTATG ACGGTCCTGA |
| 001 | TTAT | TCTTTGG |
| | (SEQ ID NO:39) | (SEQ ID NO:48) |
| PRO- | GATTTTCTTG GTTATGAGTT | CAGAGCTATG ACTATTCTGC |
| 002 | TGATTAT | TGATTAT |
| | (SEQ ID NO:40) | (SEQ ID NO:49) |
| PRO- | TATCATTCTT GGTATGAGAT | CAGAGCTATG ACTTTGATTT |
| 012 | GGGTT ATTAT GGTTCTACTG | TGCT |
| | TTGGTTATAT GTTTGATTAT | (SEQ ID NO:50) |
| | (SEQ ID NO:41) | |
| PRO- | GATAATTGGT TTAAGCCTTT | CAGCAGTATG ATTCTATTCC |
| 021 | TTCTGATGTT | TTAT |
| | (SEQ ID NO:42) | (SEQ ID NO:51) |
| PRO- | GTTAATCATT GGACTTATAC | CAGCAGATGT CTAATTATCC |
| 024 | TTTTGATTAT | TGAT |
| | (SEQ ID NO:43) | (SEQ ID NO:52) |
| PRO- | GGTTATTGGT ATGCTTATTT | CAGAGCTATG ACAATAATTC |
| 026 | TACTTATATT AATTATGGTT | TGATGTT |
| | ATTTTGATAAT | (SEQ ID NO:53) |
| | (SEQ ID NO:44) | |
| PRO- | ACTTGGCAGT ATTCTTATTT | CAGCAGACTA ATAATGCTCC |
| 029 | TTATTATCTT GATGGTGGTT | TGTT |
| | ATTATTTTGA TATT | (SEQ ID NO:54) |
| | (SEQ ID NO:45) | |
| PRO- | AATATGGCTT ATACTAATTA | CAGAGCTATG ACTATTTTAA |
| 054 | TCAGTATGTT AATATGCCTC | GCTT |
| | ATTTTGATTA T | (SEQ ID NO:55) |
| | (SEQ ID NO:46) | |
| PRO- | TCTATGAATT CTACTATGTAT | CAGAGCTATG ACATGTATAA |
| 055 | TGGTATCTTC GTCGTGTTCTT | TTATATT |
| | TTTGATCAT | (SEQ ID NO:56) |
| | (SEQ ID NO:47) | |

Antibodies

Natural antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable and Fc (fragment crystalline) domains. The antigen binding domains, Fab, include regions where the polypeptide sequence varies. The term F(ab′)₂ represents two Fab′ arms linked together by disulfide bonds. The central axis of the antibody is termed the Fc fragment. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1).

The variable domains of each pair of light and heavy chains form the antigen-binding site. The domains on the light and heavy chains have the same general structure and each domain comprises four framework regions, whose sequences are relatively conserved, joined by three hypervariable domains known as complementarity determining regions (CDR1-3). These domains contribute specificity and affinity of the antigen-binding site.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

The term “antibody” or “molecule having the antigen-binding portion of an antibody” refers to an immunoglobulin molecule able to bind to a specific epitope on an antigen, and which may be comprised of a polyclonal mixture, or be monoclonal in nature. Antibodies may be entire immunoglobulins or fragments thereof derived from natural sources, or from recombinant sources. An antibody according to the present invention may exist in a variety of forms including, for example, whole antibody, an antibody fragment, or another immunologically active fragment thereof, such as a complementarity determining region. Similarly, the antibody may be an antibody fragment having functional antigen-binding domains, that is, heavy and light chain variable domains. The antibody fragment may also exist in a form selected from the group consisting of: Fv, Fab F(ab)₂, scFv (single chain Fv), dAb (single domain antibody), bi-specific antibodies, diabodies and triabodies.

Included within the scope of the invention are chimeric antibodies; human and humanized antibodies; single domain antibodies, recombinant and engineered antibodies, and fragments thereof. Furthermore, the DNA encoding the variable region of the antibody can be inserted into the DNA encoding other antibodies to produce chimeric antibodies (see, for example, U.S. Pat. No. 4,816,567). Single chain antibodies fall within the scope of the present invention. Single chain antibodies can be single chain composite polypeptides having antigen binding capabilities and comprising amino acid sequences homologous or analogous to the variable regions of an immunoglobulin light and heavy chain (linked V_{H}-V_{L} or single chain Fv (ScFv)). Both V_{H} and V_{L} may copy natural monoclonal antibody sequences or one or both of the chains may comprise a CDR-FR construct of the type described in U.S. Pat. No. 5,091,513, the entire contents of which axe incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are incorporated herein by reference.

Additionally, CDR grafting may be performed to alter certain properties of the antibody molecule including affinity or specificity. A non-limiting example of CDR grafting is disclosed in U.S. Pat. No. 5,225,539.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab miniantibodies (see WO 93/15210; U.S. Pat. No. 5,910,573; WO 96/13583; WO 96/37621, the entire contents of which are incorporated herein by reference), dimeric bispecific miniantibodies (see Muller, et al, 1998 FEBS Letters, 432:45-49) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The term “Fc” as used herein is meant as that portion of an immunoglobulin molecule (Fragment crystallizable) that mediates phagocytosis, triggers inflammation and targets Ig to particular tissues; the Fc portion is also important in complement activation.

In one embodiment of the invention, a chimera comprising a fusion of the extracellular domain of the RPTK and an immunoglobulin constant domain can be constructed useful for assaying for ligands for the receptor and for screening for antibodies and fragments thereof.

The “extracellular domain” when used herein refers to the polypeptide sequence of the FGFR3 disclosed herein which are normally positioned to the outside of the cell. The extracellular domain encompasses polypeptide sequences in which part of or all of the adjacent (C-terminal) hydrophobic transmembrane and intracellular sequences of the mature FGFR3 have been deleted. Thus, the extracellular domain-containing polypeptide can comprise the extracellular domain and a part of the transmembrane domain. Alternatively, in the preferred embodiment, the polypeptide comprises only the extracellular domain of the FGFR3. The truncated extracellular domain is generally soluble. The skilled practitioner can readily determine the extracellular and transmembrane domains of the FGFR3 by aligning it with known RPTK (receptor protein tyrosine kinases) amino acid sequences for which these domains have been delineated. Alternatively, the hydrophobic transmembrane domain can be readily delineated based on a hydrophobicity plot of the polypeptide sequence. The extracellular domain is N-terminal to the transmembrane domain.

The term “epitope” is meant to refer to that portion of any molecule capable of being bound by an antibody or a fragment thereof, which can also be recognized by that antibody. Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody, which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies, which may be evoked by other antigens.

A “neutralizing antibody” as used herein refers to a molecule having an antigen-binding site to a specific receptor capable of reducing or inhibiting (blocking) activity or signaling through a receptor, as determined by in vivo or in vitro assays, as per the specification.

A “monoclonal antibody” or “mAb” is a substantially homogeneous population of antibodies to a specific antigen. mAbs may be obtained by methods known to those skilled in the art. See, for example Kohler and Milstein, Nature, 256(5517):495-497 (1975); U.S. Pat. No. 4,376,110; Ausubel, et al (Eds), Current Protocols in Molecular Biology, John Wiley & Sons, Inc. (New York) (1987-1999); Harlow, et al, Antibodies: A Laboratory Manual, CSHL (Cold Spring Harbor, N.Y.) (1988); and Colligan, et al (eds.), Current Protocols in Immunology, John Wiley & Sons, Inc. (New York) (1992-2000), the contents of which references are incorporated entirely herein by reference. The mAbs of the present invention may be of any immunoglobulin class including IgG, IgM, IgE, IgA, and any subclass thereof. A hybridoma producing a mAb may be cultivated in vitro or in vivo. High titers of mAbs can be obtained by in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Better et al, Science 240d (4855):1041-1043 (1988); Cabilly et al, PNAS USA 81 (11) 3273-7 (1984); Liu et al, PNAS USA, 84(10):3439-3443 (1987); Morrison et al., PNAS USA 81(21):6851-6855 (1984); Boulianne, et al, Nature 312(5995):643-646 (1984); Neuberger et al, Nature 314(6008):268-270 (1985); Cabilly et al., European Patent Applications 125023, 171496, 173494, 184187, 173494, International Patent Applications WO 86/01533, WO 97/02671, WO 90/07861, WO 92/22653 and U.S. Pat. Nos. 5,693,762, 5,693,761, 5,585,089, 5,530,101 and 5,225,539). These references are hereby incorporated by reference.

Besides the conventional method of raising antibodies in vivo, antibodies can be generated in vitro using phage display technology. Such a production of recombinant antibodies is much faster compared to conventional antibody production and they can be generated against an enormous number of antigens. In contrast, in the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Such a library can be made in several ways: One can generate a synthetic repertoire by cloning synthetic CDR3 regions in a pool of heavy chain germ line genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Pharmacology

The present invention also contemplates pharmaceutical formulations, both for veterinary and for human medical use, which comprise as the active agent one or more of the molecules having specificity and affinity to FGFR3, the molecule inducing apoptosis of myeloma cells for the manufacture of a medicament for the treatment or prophylaxis of the conditions variously described herein.

In such pharmaceutical and medicament formulations, the active agent preferably is utilized together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

Typically, the molecules of the present invention comprising the antigen binding portion of an antibody or comprising another polypeptide including a peptidomimetic, antagonistic ligand or soluble receptor or an organic molecule or polynucleotide will be suspended in a sterile saline solution for therapeutic uses. The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly (ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles (Saltzman et al, Biophys. J, 55:163 (1989); Sherwood, et al., Biotechnology, 10(11): 1446-9 (1992)). Other solid dosage forms are described in Ansel et al, Pharmaceutical Dosage Forms and Drug Delivery Systems, 5th Ed. (Lea & Febiger 1990) and Gennaro (ed.), Remington's Pharmaceutical Sciences, 18^{th} Ed. (Mack Publishing Co., 1990).

The pharmaceutical composition of this invention may be administered by any suitable means, such as orally, topically, intranasally, subcutaneously, intramuscularly, intravenously, intra-arterially, intraarticulary, intralesionally or parenterally. Ordinarily, intravenous (i.v.), intraarticular, topical or parenteral administration will be preferred.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

Although an appropriate dosage of a molecule of the invention varies depending on the administration route, type of molecule (polypeptide, polynucleotide, organic molecule etc.) age, body weight, sex, or conditions of the patient, and should be determined by the physician in the end, in the case of oral administration, the daily dosage can generally be between about 0.01 mg to about 500 mg, preferably about 0.01 mg to about 50 mg, more preferably about 0.1 mg to about 10 mg, per kg body weight. In the case of parenteral administration, the daily dosage can generally be between about 0.001 mg to about 100 mg, preferably about 0.001 mg to about 10 mg, more preferably about 0.01 mg to about 1 mg, per kg body weight. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those in the art. In addition, if desired, the composition can contain minor amounts of auxiliary substances such as wetting or emulsifying agents, pH buffering agents.

The combined treatment of one or more of the molecules of the invention with an anti-inflammatory drug such as methotrexate or glucocorticoids may provide a more efficient treatment for inhibiting FGFR3 activity. In one embodiment, the pharmaceutical composition comprises the antibody, an anti-inflammatory drug and a pharmaceutically acceptable carrier.

Polynucleotides

The term “nucleic acid” and “polynucleotides” refers to molecules such as deoxyribonucleic acid (DNA), and, where appropriate, ribonucleic acid (RNA). The term should also be understood to include, as equivalents, analogs of RNA or DNA made from nucleotide analogs, and, as applicable to the embodiment being described, single (sense or antisense) and double-stranded polynucleotides.

Within the scope of the present invention is a nucleic acid molecule encoding an anti-FGFR3 antibody useful for the preparation of a medicament for the treatment of multiple myeloma. The nucleic acid molecule contains a nucleotide sequence having at least 75% sequence identity, preferably about 90%, and more preferably about 95% identity to the above encoding nucleotide sequence set forth in any one of SEQ ID NOS: 57-74, as would be well understood by those of skill in the art. In the hypervariable regions of the heavy chain and light chain, the nucleic acid molecule contains a nucleotide sequence having at least 50% sequence identity, preferably about 70% and more preferably about 80% identity to the molecules set forth in any one of SEQ ID NOs: 39-56.

The invention also provides nucleic acids that hybridize under high stringency conditions to polynucleotides set forth in any one of SEQ ID NOs: 57-74 or the complement thereof. As used herein, highly stringent conditions are those which are tolerant of up to about 5%-25% sequence divergence, preferably about 5%-15%. Without limitation, examples of highly stringent (−10° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5×Denhardt's reagent, 0.5% SDS, 100 μg/ml denatured, fragmented salmon sperm DNA at an appropriate incubation temperature Ti. See generally Sambrook et al., Molecular Cloning: A Laboratory Manual, 2d edition, Cold Spring Harbor Press (1989)) for suitable high stringency conditions.

Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA: DNA hybrids and 10-15° C. below Tm for DNA: RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA: DNA hybrids using the equation of Meinkoth and Wahl (Anal. Biochem. 138 (2): 267-84 (1984)), as

Tm=81.5° C.+16.6(log M)+0.41(% GC)−0.61(% form)−500/L

and for DNA:RNA hybrids, as

Tm=79.8° C.+18.5(log M)+0.58(% GC)−11.8(% GC)²−0.56(% form)−820/L

where
M, molarity of monovalent cations, 0.01-0.4 M NaCl,
% GC, percentage of G and C nucleotides in DNA, 30%-75%,
% form, percentage formamide in hybridization solution, and
L, length hybrid in base pairs.

Tm is reduced by 0.5-1.5° C. (an average of 1° C. can be used for ease of calculation) for each 1% mismatching. The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired high stringency. The equations for Tm can be used to estimate the appropriate Ti for the final wash, or the Tm of the perfect duplex can be determined experimentally and Ti then adjusted accordingly.

The invention also provides for conservative amino acid variants of the molecules. Variants according to the invention also may be made that conserve the overall molecular structure of the encoded proteins. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

Many therapeutic human proteins suffer from short half life and low stability in the circulation, and therefore require the use of high doses to maintain therapeutic efficacy. PEGylation is a method for the covalent attachment of PEG to proteins (reviewed in Greenwald et al. (2003) Advanced Drug Delivery Reviews 55 217-250). PEG (Poly ethylene glycol) is a unique polymer which dissolves in organic solvents as well as in water; it is non-toxic and eliminated by a combination of renal and hepatic pathways thus making it ideal to employ in pharmaceutical applications. A PEGylated protein usually has significantly increased half life in the blood circulation, reduced immunogenicity and antigenicity while retaining its bioactivity.

Early work on proteins often utilized PEG of Mw 5000. However, fewer strands of PEG of higher Mw are also employed e.g. PEG of Mw 20,000 or 40,000.

The invention therefore also provides for PEGylated versions of the molecules of the invention. Specifically, the invention encompasses PEGylated monoclonal antibodies or fragments thereof having specificity and affinity for FGFR3 that have increased in vivo half-lives allowing to reduce the dosage and/or frequency of administration of said antibodies or fragments thereof to a subject.

The molecules may be PEGylated by any of the PEGylation methods which are well known in the art (Lee et al. (1999) Bioconjugate Chem. 10 973-981) using PEG molecules of different molecular weights ranging from Mw 5,000 to Mw 40,000, but preferably using PEG molecules of Mw 5,000 to 20,000.

In order to allow PRGylation with minimal hindrance to the bioactivity of the molecule the PEG moiety may be appended at the N-terminus of the molecule. For that purpose the scFv molecule of the invention (SEQ ID: 37) was generated wherein the amino acid at the N-terminus (position 2) is serine instead of leucine, thus allowing targeted PEGylation.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Methods

Cell Lines and Tissue Culture

Non-transformed rat chondrocyte cell line expressing PGFR3 in an inducible manner (RCJ-FGFR3) has been described previously (Rauchenberger R. et al. J. Biol. Chem. 2003; 278:38194-205). Cells were maintained in α-Minimum Essential Media supplemented with 15% fetal calf serum (FCS), 2 mM L-Glutamine, 100 U/ml penicillin, 100 μg/ml streptomycin, 600 μg/ml G418 (Gibco BRL, Ontario, Canada), 2 μg/ml Tetracyclin (Sigma, Ontario, Canada), and 50 μg/ml HygromycinB (Gibco BRL). FGFR3 expression was induced by removal of tetracyclin and serum starvation. The mouse myeloid progenitor cell line (FDCP-1) was transfected with full length FGFR1 (FDCP-FGFR1), FGFR2 (FDCP-FGFR2), FGFR3 (FDCP-FGFR3) or FGFR3^{S249C} mutant cDNAs and cultured in Iscove's medium (GibcoBRL) with 10% FCS, 100 μg/ml penicillin, 100 μg/ml streptomycin, 10 ng/ml FGF and 5 μg/ml heparin (Sigma). Human myeloma cell lines (UTMC2, H929, KMS11, KMS18, 8226) were maintained in Iscove's Modified Dulbecco's Medium (IMDM) supplemented with 2.5% FCS and penicillin-streptomycin (Hyclone, Logan, Utah). B9 cells expressing WT FGFR3 (B9-FGFR3^{WT}), FGFR3-F384L (B9-FGFR3^{F384L}), FGFR3-Y373C (B9-FGFR3^{Y373C}), FGFR3-G394D (B9-FGFR3^{G394D}) have been previously described (Plowright E E, et al. Blood. 2000; 95:992-998; Trudel S, et al. Blood. 2005; 105:2941-8). These were maintained in IMDM supplemented with 5% FCS, penicillin-streptomycin and 1% IL-6 conditioned medium. Bone marrow stroma cells (BMSCs) were derived from bone marrow (BM) specimens obtained from MM patients and prepared as previously described (Hideshima T, et al. Blood 2000; 96:2943-2950). BMSCs were grown on 6 well plates until confluent and were then irradiated with 20 Gy for the apoptosis studies described below.

Immunoprecipitation and Immunoblotting

Cells were lysed in lysis buffer (50 mM Tris/HCl, pH 8.0, 150 mM NaCl₂, 0.1 mM ZnCl₂, 0.5% Nonidet NP-40, 1 mg/ml, complete protease inhibitor mix (Roche Molecular Biochemicals, Mannheim, Germany)), and clarified by centrifugation at 12,000×g for 15 minutes. The lysates were subjected to immunoprecipitation for 16 hours at 4° C. with anti-FGFR3 (C15) and analyzed by 7.5% sodium dodecyl-polyacrylamide gel electrophoresis (SDS-PAGE) and Western blot with anti-phosphotyrosine (4G10 from R&D). Protein bands were visualized using secondary antibodies coupled to horseradish peroxidase and the ECL kit from Pierce according to the manufacturer's instructions.

Viability Assay

Cell viability was assessed by 3-(4,5-dimethylthiazol)-2,5-diphenyl tetrazolium (MTT) or (2,3-bis(2-methoxy-4-nitro-5-sulphophenyl)-5-[(phenylamino) carbonyl]-2H-tetrazolium hydroxide (XTT) dye absorbance where indicated. Cells were seeded in 96-well plates at a density of 20,000 (FDCP-1 cells), 5,000 (B9 cells) or 25,000 cells (MM cell lines) per well in culture medium. Cells were incubated in the absence or presence of one of the following cytokines: 10 ng/ml FGF9 and 5 μg/ml heparin, 1% murine IL-6, 50 ng/ml IGF-1 or 50 ng/ml human IL-6 where indicated and increasing concentrations of PRO-001, control antibody (purified human Fab) or 100 nM PD173074. Plates were incubated for 48 or 72 h at 37° C., 5% CO₂. MTT and XTT assays were performed according to the manufacturer's instruction (Boehringer Mannheim, Mannheim, Germany and Biological Industries Ltd., Israel, respectively). Each experimental condition was performed in duplicate or triplicate.

Proliferation Assay

Cell proliferation was determined by [³H]-thymidine incorporation assay. UTMC2 cells (20,000 cells/well) were incubated at 37° C. in 96-well plates in the presence of vehicle control or 5 μg/ml PRO-001. [³H]-thymidine (0.5 μCi) was added to each well for 8 h. Cells were harvested onto glass filters with an automatic cell harvester and counted by PACKARD TOP counter (CANBERPA PACKARD, Canada).

Flow Cytometric Analysis

Cells (5×10⁵) were washed in cold phosphate-buffered saline (PBS) and then incubated for 30 minutes with one of the following: PRO-001 Fab or a human Fab control antibody, rabbit anti-FGFR3 (100) or rabbit preimmune serum in PBS. The cells were then stained with PE-conjugated goat antihuman F(ab′)₂ secondary antibody or goat anti-rabbit IgG-PE for 30 minutes on ice. Flow cytometry was performed on a FACSCaliber flow cytometer (BD Biosciences, San Jose, Calif.) and analyzed using Cellquest software (Becton Dickinson). To assess the ability of FGF ligand to compete for binding, cells were incubated in the presence or absence of 30 ng/ml FGF and 5 μg/ml heparin for 30 minutes and then stained with PRO-001 Fab as describe above.

Intracellular Phospho-Protein Staining

Determination of ERK1/2 phosphorylation by flow cytometry has been described previously (Chow S, et al., Cytometry 2001; 46:72-78). Cells were serum-starved overnight and then stimulated with 30 ng/ml of aFGF and 10 μg/ml heparin for 10 minutes at 37° C. The cells were immediately fixed by adding 10% formaldehyde directly into the culture medium to obtain a final concentration of 2%. Cells were incubated in fixative for 10 min at 37° C. then on ice for an additional 2 minutes. The cells were permeabilized by adding ice-cold methanol (to a final concentration of 90%) while vortexing and incubated on ice for 30 minutes. Cells were washed with PBS plus 4% FCS, stained with anti-ERK1/2 (Cell Signaling Technology, Beverly, Mass.) for 15 minutes and then labeled with fluorescein isothiocyanate (FITC) conjugated goat anti-rabbit and anti-CD138-PE (PharMinogen, San Diego, Calif.) where indicated. Malignant cells were identified as cells that express high levels of CD138. Flow cytometry was performed on a FACSCaliber® flow cytometer (BD Biosciences, San Jose, Calif.) and analyzed using Cellquest® software (Becton Dickinson).

Apoptosis Analysis

For studies of apoptosis, cells were seeded at an initial density of 2.5×10⁵/ml in 6 well plates coated with BMSCs and supplemented with control (vehicle or antibody) or 5 μg/ml PRO-001 and cultured for 48 h. Apoptosis was determined by Annexin V staining (Boehringer Mannheim, Indianapolis, Ind.) and analyzed by flow cytometry. Annexin V is a protein that binds specifically to phosphotidyl-serine in the cell membrane. Binding occurs once the membrane has started to break down and the phospholipids are released into the extracellular media.

Primary Patient Samples

Patients identified for the study were determined to possess a t(4; 14) translocation by fluorescence in situ hybridization (FISH). Expression of FGFR3 was confirmed by flow cytometry as previously described (Chesi M, et al. Blood. 2001; 97:729-736). Briefly, erythrocytes were lysed and bone marrow mononuclear cells were incubated on ice for 30 minutes with rabbit anti-FGFR3 (H100) or rabbit pre-immune serum. The cells were washed and then stained with FITC-conjugated goat anti-rabbit IgG and mouse anti-CD138-PE to identify MM cells. The samples were then analyzed by flow cytometry.

All t(4; 14) positive samples were further analyzed for the presence of FGFR3 mutations. Four pairs of primers were designed to amplify the regions of FGFR3 containing codons of the extracellular (EC) domain, transmembrane (TM) domain tyrosine kinase (TK) domain and stop codon (SC), known hot spots for activating mutations. A first PCR reaction was performed on genomic DNA extracted from CD138 purified myeloma cells and amplicons were used for DHPLC analysis. Results were confirmed by sequence analysis of the PCR products.

For cell death analysis, mononuclear cells freshly isolated from bone marrow aspirates were separated by Ficoll-Hipaque gradient sedimentation and plated at a cell density of 5×10⁵ cells/ml in IMDM supplemented with 20% FCS, 1% glutamine, penicillin-streptomycin and 30 ng/ml aFGF and 10 μg/ml heparin. Cells were cultured in the presence of control or 5 μg/ml PRO-001 for up to 12 days. The medium, aFGF/heparin and drug were replenished every 3 days. After 3, 7 and 12 days, cells were triple stained with anti-CD38-PE, anti-CD45-CyChrome (PharMinogen) and FITC-conjugated Annexin V or labeled with anti-CD138-PE and FITC-conjugated annexin V. Controls included unstained cells, isotype control stained cells, and single-stained cells. Malignant plasma cells were defined as cells that express CD138 or high levels of CD38 and no or low levels of CD45 (CD38⁺⁺/CD45⁻). Samples were analyzed by FACScan analysis using Cellquest software. Bone marrow aspirates were obtained by consent under an IRB-approved protocol.

Xenograft Mouse Model

FDCP-FGFP3^{S249C} cells were washed 3 times in PBS then resuspended at 2×10⁶ cells/200 μl PBS. The cells were injected subcutaneously (S.C.) to CD1 nude adult females (Harlan, Laboratories, Israel) with a 25 G needle at one or both mouse flanks. Treatment was initiated one week post cell inoculation at which time mice were randomized to receive PRO-001 or an equal volume of PBS alone. Dosing was preformed twice weekly by intraperitoneal (I.P.) injection for 3 weeks. Mice were followed every 2-4 days and developing tumors were measured at 3 dimensions using a caliper. Tumor volume was estimated by multiplying these 3 values.

PEGylation

RPO-001Ser scFv was diluted 5 times in PBS to 1 mg/ml and was oxidized at room temperature with 10 fold excess periodate over 10 minutes. The reaction was terminated by the addition of 10 fold excess diaminopropanol over the oxidizing agent for a further 15 minutes. The oxidized material was dialyzed 2 hours at room temperature against PBS then the pH was lowered by further dialysis at room temperature against 50 mM NaOAc pH 5.3. mPEG-HZ-5K and mPEG-HZ-20K (purchased from IDB) were dissolved in acetate pH 5.3 and added to oxidized PRO59scSer at 10 and 2.5 fold molar excess, respectively. mPEG-HZ-40K (purchased from Nektar), dissolved in water was added 1.3 equivalents to the oxidized single chain. The reaction products were analyzed 24 hours later by coomassie stained SDS-PAGE.

### Example 1

### Blocking Activity of PRO-001 and Selectivity for FGFR3

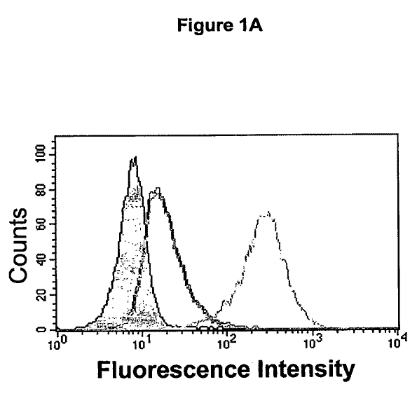

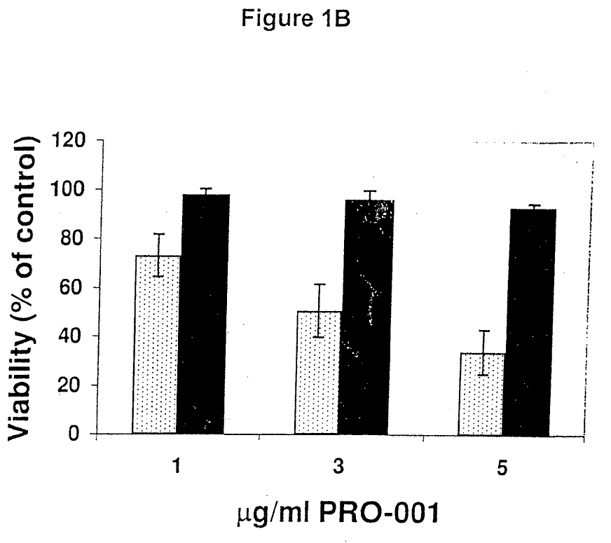

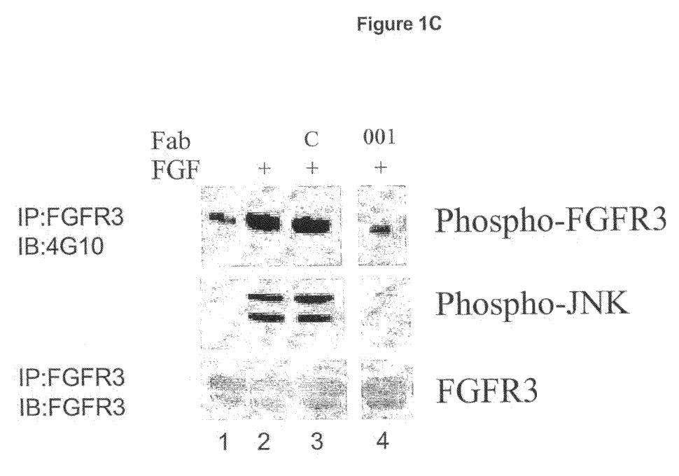

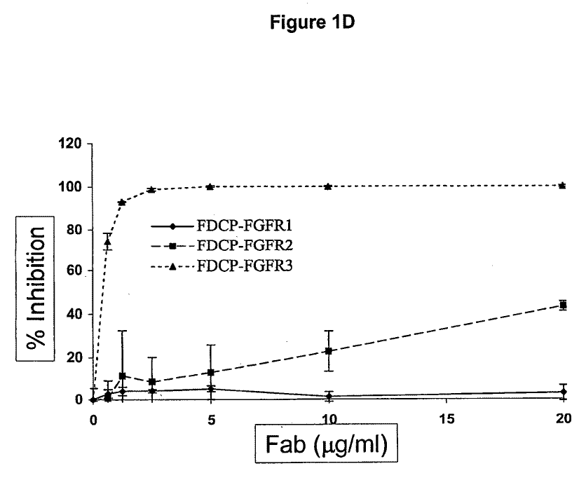

The human anti-FGFR3 Fab PRO-001 was isolated from the Hu-CAL®-Fab-1 human combinatorial library using a differential whole cell panning approach (Rauchenberger R, et al. J Biol. Chem. 2003; 278:38194-205). FACS analysis revealed that PRO-001 Fab binds to WT FGFR3 and that binding to B9-FGFR3^{WT} cells can be reduced by addition of FGF, supporting the notion that PRO-001 and FGF share a common epitope (FIG. 1A). FIG. 1B shows that PRO-001 inhibits growth of FGF stimulated B9-FGFR3^{WT} cells. The growth inhibition is dose dependent. One microgram antibody per millilitre (1 μg/ml) inhibits growth by about 25% while 5 μg/ml antibody inhibits growth by more than 60%. Moreover, PRO-001 also inhibits the FGF-stimulated growth of B9 cells expressing the FGFR3 mutant F384L (a non-transforming polymorph of FGFR3), as well as the FGF-stimulated growth of cells expressing G394D and Y373C-FGFR3 (constitutively activated FGFR3 mutants identified in MM patients) in a dose-dependent manner with an IC₅₀ of approximately 3 μg/ml consistent with its ability to inhibit FGF binding.

To confirm that PRO-001 inhibits the kinase activity of FGFR3, we tested the effect of PRO-001 on ligand stimulated receptor phosphorylation in RCJ cells transfected with WT FGFR3 (RCJ-FGFR3). Anti-phosphotyrosine immunoblots revealed enhanced autophosphorylation of FGFR3 upon ligand stimulation that was inhibited by PRO-001 but not by the control Fab (FIG. 1C). Inhibition of FGFR3 activation was associated with reduction in downstream JNK phosphorylation. To confirm the specificity and blocking activity of PRO-001 in a cell-based assay, we tested the activity of PRO-001 against FGFR 1-3 expressing FDCP-1 cell lines. Cell growth of FDCP-1 is normally dependent on the presence of IL-3. However, IL-3 can be substituted by FGF ligand in cells expressing the cognate RTK. FGF stimulated proliferation of FDCP-FGFR3 cells was potently inhibited by PRO-001, with IC₅₀ (concentration that inhibits 50% of the cells) of 0.5 μg/ml (FIG. 1D). In contrast, the proliferation of FDCP-1 cells expressing FGFR1 or FGFR2 was unaffected up to 10 fold higher concentrations. Thus PRO-001 is a highly specific and potent inhibitor of FGFR3.

### Example 2

### Anti-FGFR3 Inhibits Viability of aFGF-Stimulated UTMC2 Human Myeloma Cells

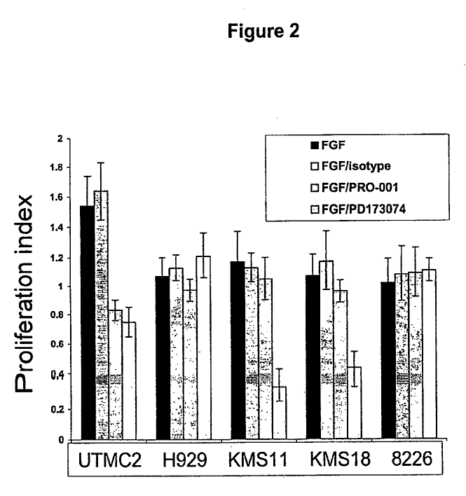

PRO-001 was tested against t (4; 14) myeloma cell lines expressing FGFR3: UTMC2 cells—expressing WT FGFR3, and H929 cells—expressing WT FGFR3 but harboring a downstream activating mutation of N-Ras. Cell growth in the presence of FGF and PRO-001 (5 μg/ml), control antibody (isotype) or 100 nM PD173074 was determined by MTT assay. Proliferation of FGF-stimulated UTMC2 cells was significantly inhibited by PRO-001 (FIG. 2). Inhibition of FGF-stimulated growth of UTMC2 by PRO-001 was comparable to that induced by PD173074 (An ATP analog which binds and inhibits the kinase domain). 8226 cells, which lack FGFR3 expression and H929 cells were resistant to both PRO-001 and PD173074, indicating that both reagents act upstream of Ras and target selectively FGFR3.

PRO-001 failed to inhibit the viability of KMS11 (FGFR3-Y373C) and KMS18 (FGFR3-G384D), cells that express mutant FGFR3 and grow independent of FGF.

### Example 3

### Anti-FGFR3 Inhibits Downstream ERK1/2 Phosphorylation of aFGF-Stimulated UTMC2 Human Myeloma Cells

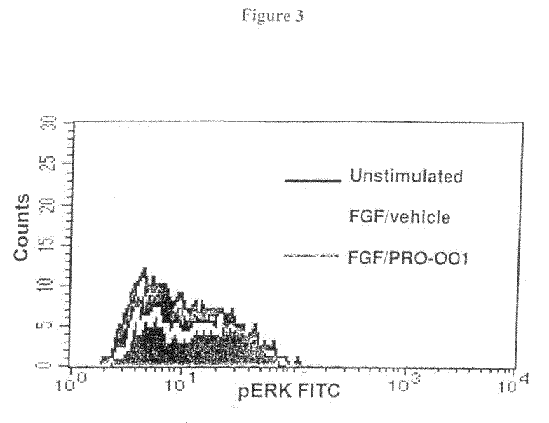

FIG. 3 shows the inhibition of Extracellular signal-regulated protein kinase (ERK) 1/2 phosphorylation upon incubation of aFGF-stimulated UTMC2 cells with the anti-FGFR3 antibody of the present invention, as detected by flow cytometry. The levels of phosphorylated ERK return to those of unstimulated cells upon incubation with the anti-FGFR3 antibody of the invention.

### Example 4

### IL-6 and IGF-I do not Confer Resistance to Anti-FGFR3

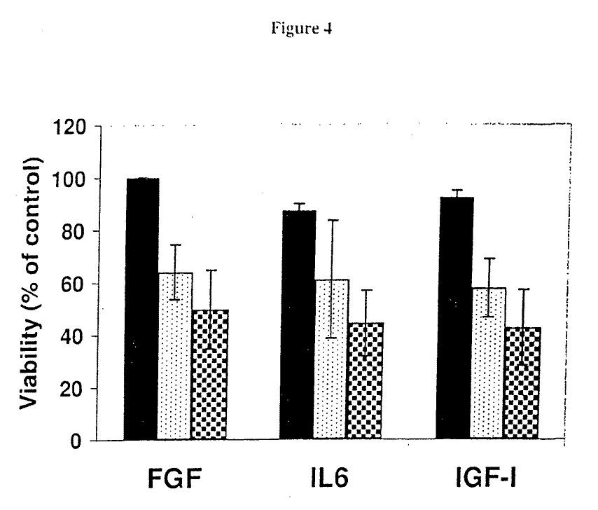

FIG. 4 shows viability of cells stimulated with FGF9 (30 ng/ml), IL6 (Song/ml), or IGF-1 (Song/ml), and treated with the anti-FGFR3 antibody. IL6 and IGF-1 stimulate the myeloma cells, which remain sensitive to treatment with the anti-FGFR3 antibody. These results demonstrate that paracrine factors known to confer drug resistance fail to overcome the potential anti-tumour effects of FGFR inhibition. Cells were treated with an FGFR inhibitor, PD173074, as control.

### Example 5

### Anti-FGFR3 Induces Apoptosis of UTMC2 Cells Co-Cultured with Bone Marrow Stroma Cells

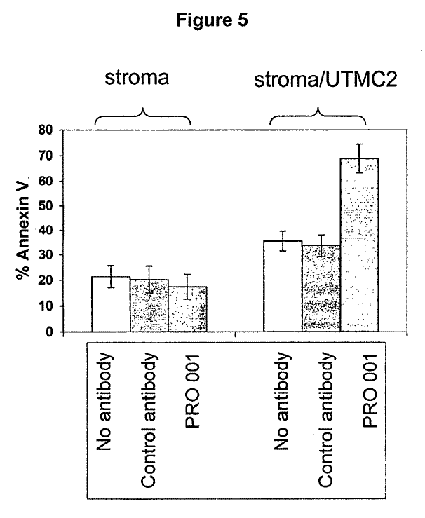

Anti-FGFR3 induces a high level of apoptosis of the UTMC2 cells when co cultured with bone marrow stroma cells, BMSC, thus mimicking the milieu of the myeloma cells (FIG. 5). The antibody had no direct toxicity on the BMSC. These data are consistent with previous studies using FGFR3 small molecule kinase inhibitors.

### Example 6

### Anti-FGFR3 Induces Apoptosis of FGFR3 Expressing Primary Myeloma Cells

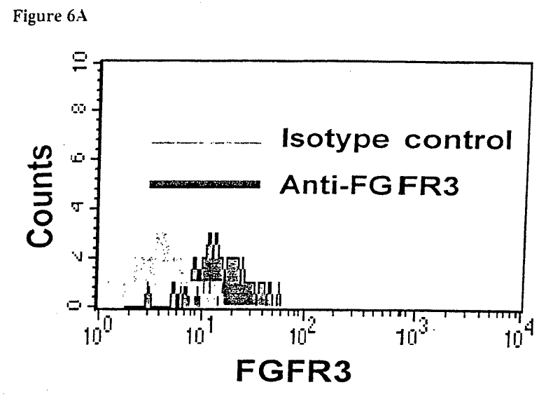

The next experiments were designed to examine the effect of PRO-001 on primary human MM cells. Bone marrow samples were obtained from 10 patients, 5 of which were previously identified by FISH as t (4; 14) positive. The characteristics of the samples including measurement of FGFR3 expression by flow cytometry and FGFR3 genotype are summarized in Table I. Of the t (4; 14) positive samples tested, CD138 myeloma cells showed surface expression of FGFR3 and no mutations of FGFR3 were identified. FIG. 6A shows that cells expressing FGFR3 are identified by the anti-FGFR3 antibody (black line) and not by an isotype control (grey line). FIG. 6B shows that PRO-001 blocked FGF-induced ERK phosphorylation in myeloma cells (dark grey) when compared to cells exposed to FGF (light grey). Unstimulated cells are shown for comparison (dark area).

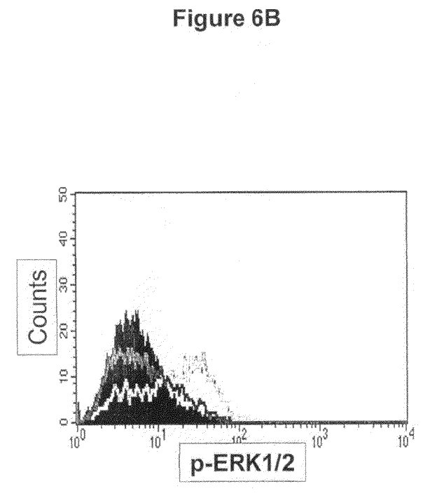

Finally, the mononuclear cell fractions isolated from fresh bone marrow samples were incubated with 5 μg/ml PRO-001 or isotype control, and apoptosis was determined by annexin V staining of CD38⁺⁺/CD45⁻ cells and loss of surface CD138 expression. All FGFR3-expressing myeloma samples displayed potent apoptotic responses to PRO-001 when compared to the control antibody (FIG. 7 shows a representative experiment.). Further, the cytotoxic effect was selective in that none of the t (4; 14) negative samples demonstrated increased apoptosis in response to PRO-001.

TABLE I
Summary of expression of FGFR3 on primary MM
cells in relation to sensitivity to PRO-001

| | FGFR3 | | | | |
|---|---|---|---|---|---|
| | (flow | FGFR3 | | % Annexin V | |
| Pa- | cytom- | geno- | % Annexin V | PRO-001 | % Increase |
| tient | etry) | type | Control | (5 μg/ml) | Annexin V |
| 1 | N/D | WT | 8.0 | 47.6 | 36.5 |
| 2 | + | WT | 12.4 | 35.7 | 20.4 |
| 3 | ++ | WT | 35.3 | 67.5 | 32.2 |
| 4 | ++ | WT | 18.2 | 98.3 | 80.0 |
| 5 | +++ | WT | 10.0 | 28.3 | 18.3 |
| 6 | − | N/D | 5.0 | 10.6 | 5.6 |
| 7 | − | N/D | 12.9 | 9.9 | −3.0 |
| 8 | − | N/D | 23.0 | 30.0 | 7.0 |
| 9 | − | N/D | 10.8 | 9.5 | −1.3 |
| 10 | − | N/D | 22.3 | 24.3 | 2.0 |

Key: FGFR3 expression on CD138 primary MM cells was analyzed by flow cytometry 20 and the fluorescence was expressed as follows: +, weak; ++ intermediate; +++ strong; − absent. CD138 selected cells were screened for FGFR3 mutations. WT denotes wild-type status and N/D indicates not determined.

### Example 7

### SCID Mouse Tumor Model

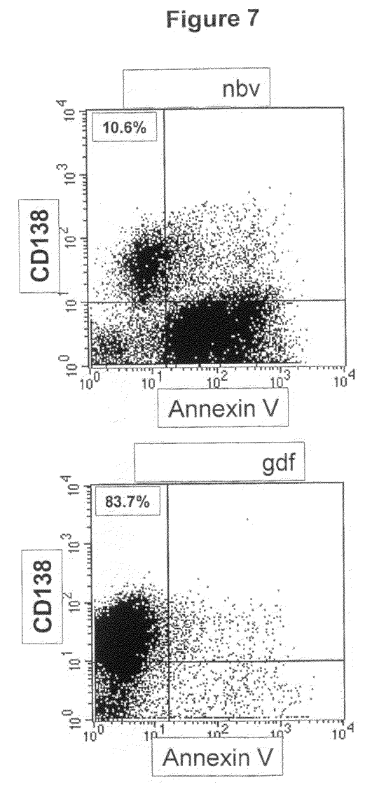

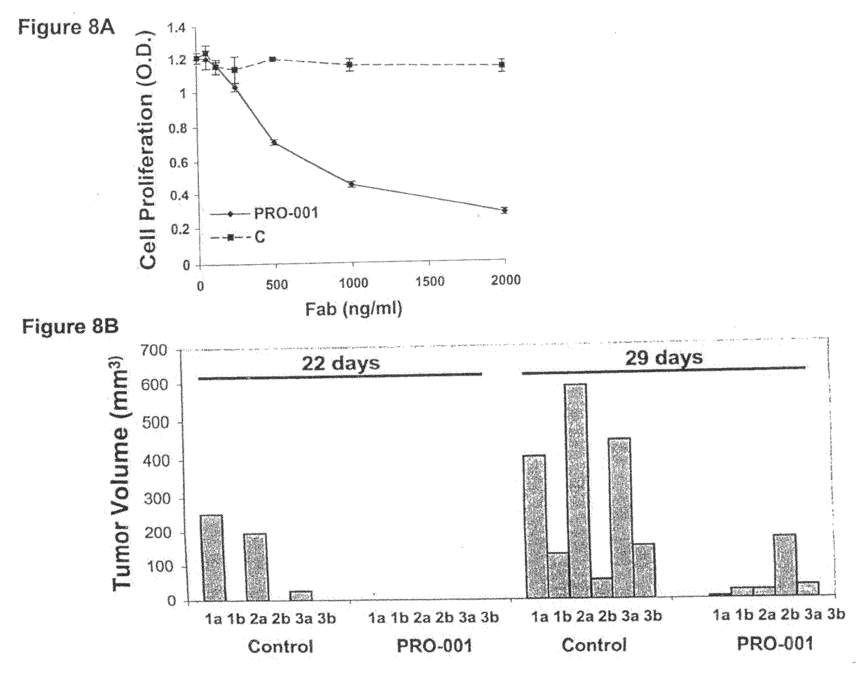

In order to evaluate the potential anti-tumor effects of the anti-FGFR3 in vivo, we used an animal model comprising nude mice injected with FDCP cells that express the constitutive mutant FGFR3^{S249C} (FDCP-FGFR3^{S249C}). FDCP-FGFR3^{S249C} proliferate in the absence of IL-3 and FGF and rapidly (within 2-3 weeks) form tumors upon injection to nude mice. PRO-001 efficiently blocked FGF-independent proliferation of FDCP-FGFR3^{S249C} in vitro (FIG. 8A).

Nude mice were injected subcutaneously at 2 locations; one on each flank, with 2×10⁶ FDCP-FGFR3^{S249C} cells each. One week post cell injection, mice were treated with PRO-001 Fab. During the first week of treatment, mice received a relatively high dose of about 1 mg Fab per mouse in order to saturate FGFR3. This was followed by slightly reduced doses during the following 12 days of Fab delivery (Table II). Mice were treated every 3 days on average, as we found no significant difference in efficacy of this schedule in comparison to daily injections (not shown). Four weeks post cell injection, PRO-001 dramatically reduced tumor growth to 10% in average of that in the control mice (FIG. 8B). No major toxicities or significant weight loss was observed over the treatment period.

TABLE II
Schedule and dosing of PRO-001 Fab or PBS administration

| | Days After FDCP-FGFR3^{S249C} Cell Injection | | | | | | |
|---|---|---|---|---|---|---|---|
| | 7 | 10 | 13 | 16 | 20 | 23 | 25 |
| PRO-001 (μg) | 400 | 400 | 275 | 275 | 275 | 275 | 275 |

The present invention is exemplified by certain animal disease models. These models are intended as a non-limitative example used for illustrative purposes of the principles of the present invention.

### Example 8

### PEGylation of PRO-001 scFv

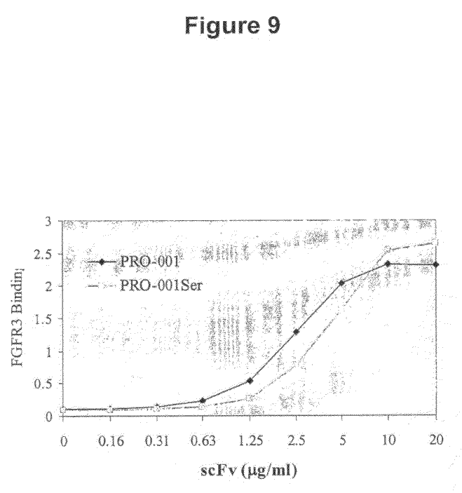

A PEG moiety was appended at the amino-terminus of the single chain antibody of the invention (PRO-001 scFv) through a serine residue. A scFv (SEQ ID No: 37) was generated having the amino acid serine at position 2 to allow PEGylation. PRO-001 scFv with a serine at the N-terminus was generated by PCR and confirmed by sequencing as previously describe (WO 02/102972). Briefly, the inclusion bodies were washed in PBS, PBS+0.1% triton, and 3M urea. The washed pellet was dissolved in PBS+5M urea, GSH/GSSG (0.5 mM each) redox potential was added and then gradually dialyzed against urea step gradient. The binding activity of the refolded PRO-001ser scFv to FGFR3 was compared by ELISA showing similar activity as the parental single chain (FIG. 9).

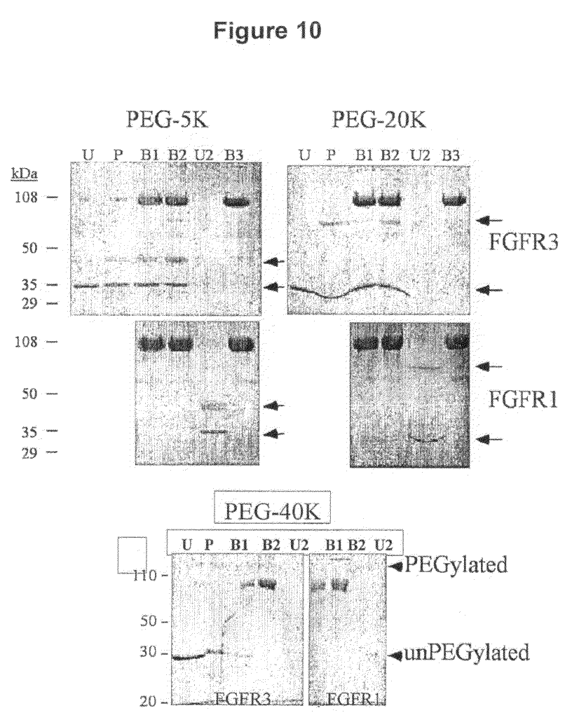

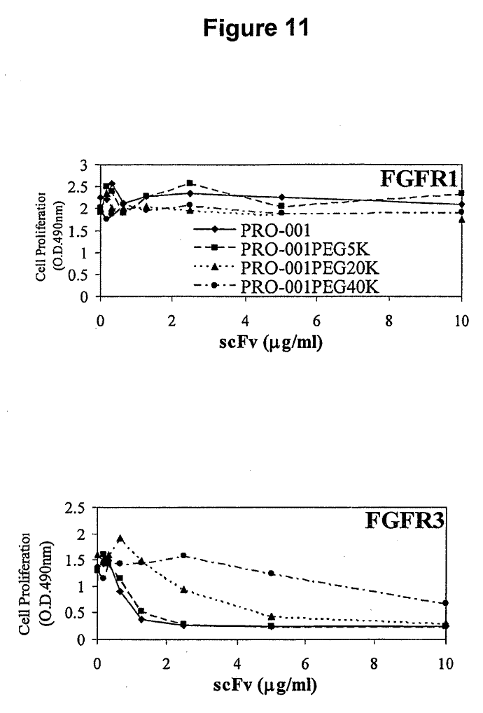

PEGylation was performed as describe in the METHODS section. Analysis of the reaction products by coomassie stained SDS-PAGE revealed that approximately 50% of the single chain was conjugated to either mPEG-HZ-5K, mPEG-HZ-20K or mPEG-HZ-40K (FIG. 10). We next examined the activity of the PEGylated single chain by incubating the reaction mix on FGFR3ex/Fc-protein A sepharose. The unbound fraction was collected and subjected to 2 more cycles with FGFR3ex/Fc-protein A sepharose. The bound material of each cycle and unbound fraction of the last one were analyzed by coomassie staining demonstrating that both the unmodified as well as the PEGylated single chain bound to the FGFR3 as both types were present only in the bound but not in the unbound fraction. Reciprocal distribution was obtained upon fractionation with FGFR1ex/Fc-protein A sepharose demonstrating the specific recognition by the PEGylated PRO-001Ser scFv. To determine the relative activity of the PEGylated PRO-001Ser scFv the PEGylation reaction products were applied to Q-sepharose anion exchanger and fractions containing predominantly PEGylated single chain were obtained. PEGylated PRO-001 was added at increasing levels to FDCP-FGFR3 or FDCP-FGFR1 cells and cell proliferation was measured. PRO-001 PEGylated with mPEG-HZ-5K retained full FGFR3 neutralizing activity (FIG. 11). Conjugation to mPEG-HZ-20K reduced the antibody activity by 5 fold and to mPEG-HZ-40K by approximately 40 fold.

1. A method for the prevention, attenuation or treatment of a B-cell malignancy comprising administering to a subject in need thereof a pharmaceutical composition comprising a therapeutically effective amount of a molecule comprising the antigen-binding portion of an isolated antibody having specificity and affinity for FGFR3, the molecule inducing apoptosis of a myeloma cell, and a pharmaceutically acceptable carrier.

2. The method of claim 1 wherein the B-cell malignancy is multiple myeloma.

3. The method according to claim 2 wherein the myeloma cell is expressing wild type FGFR3.

4. The method according to claim 1 wherein said molecule comprising the antigen-binding portion of an antibody is a molecule having specificity and affinity for the extracellular domain of FGFR3.

5. The method according to claim 1 wherein the molecule comprising the antigen-binding portion of an isolated antibody is selected from a polyclonal antibody, a monoclonal antibody, a chimeric antibody, a single domain antibody, a recombinant antibody and fragments thereof.

6. The method according to claim 5 wherein said molecule comprising the antigen-binding portion of an antibody which has a specific affinity for FGFR3 is a monoclonal antibody or a proteolytic fragment thereof.

7. The method according to claim 6 wherein said monoclonal antibody or proteolytic fragment thereof is a Fab fragment.

8. The method according to claim 5 wherein the molecule comprising the antigen-binding portion of an isolated antibody is a recombinant antibody.

9. The method according to claim 8 wherein the molecule comprising the antigen-binding portion of an isolated antibody is a recombinant Fab antibody fragment.

10. The method according to claim 8 wherein the molecule comprising the antigen-binding portion of an isolated antibody is a recombinant single chain antibody.

11. The method according to claim 6 wherein said molecule comprising the antigen-binding portion of an antibody comprises a VH-CDR3 region selected from a group consisting of polypeptides set forth in any one of SEQ ID NOS: 1-9 and a VL-CDR3 region selected from a group consisting of polypeptides set forth in anyone of SEQ ID NOS: 10-18; a VH region selected from a group of polypeptides set forth in anyone of SEQ ID NOS: 19-27 and a VL region selected from the group of polypeptides set forth in anyone of SEQ ID NOS: 28-36; a VH-CDR3 region encoded by a polynucleotide sequence set forth in anyone of SEQ ID NOS: 39-47 and a VL-CDR3 region selected from a group consisting of polypeptides set forth in anyone of SEQ ID NOS: 48-56; or a VH region encoded by a polynucleotide sequence selected from a group of polynucleotides set forth in anyone of SEQ ID NOS: 57-65 and a VL region encoded by a polynucleotide sequence selected from the group of polynucleotides set forth in anyone of SEQ ID NOS: 66-74.

12. The method according to claim 11 wherein said molecule comprising the antigen-binding portion of an antibody which has a specific affinity for FGFR3 comprises a VH-CDR3 region set forth in SEQ ID NO: 1 and a VL-CDR3 region set forth in SEQ ID NO: 10; a VH region set forth in SEQ ID NO: 19 and a VL region set forth in SEQ ID NO: 28; a VH-CDR3 region encoded by a polynucleotide sequence set forth in SEQ ID NO: 39 and a VL-CDR3 region set forth in SEQ ID NO:48; a VH region encoded by a polynucleotide set forth in SEQ ID NO: 57 and a VL region encoded by a polynucleotide set forth in SEQ ID NO: 66; a single chain Fv encoded by a polynucleotide having a sequence set forth in SEQ ID NOS: 37 or 38; or is PEGylated.

13.-15. (canceled)

16. The method according to claim 5 wherein said molecule comprising the antigen-binding portion of an antibody which has a specific affinity for FGFR3 is a single chain Fv set forth in SEQ ID NO: 37 or is PEGylated.

17.-25. (canceled)

26. A pharmaceutical composition for the prevention, attenuation or treatment of a B-cell malignancy comprising as an active ingredient a therapeutically effective amount of a molecule comprising the antigen-binding portion of an isolated antibody having specificity and affinity for FGFR3, the molecule inducing apoptosis of a myeloma cell, and a pharmaceutically acceptable carrier.

27. A pharmaceutical composition according to claim 26 wherein the B-cell malignancy is multiple myeloma.

28. A pharmaceutical composition according to claim 26 wherein said molecule comprising the antigen-binding portion of an antibody is a molecule having specificity and affinity for the extracellular domain of FGFR3.

29. A pharmaceutical composition according to claim 26 wherein the molecule comprising the antigen-binding portion of an isolated antibody is selected from a polyclonal antibody, a monoclonal antibody, a chimeric antibody, a. single domain antibody, a recombinant antibody and fragments thereof.

30. A pharmaceutical composition according to claim 29 wherein said molecule comprising the antigen-binding portion of an antibody which has a specific affinity for FGFR3 is a monoclonal antibody or a proteolytic fragment thereof.

31. A pharmaceutical composition according to claim 30 wherein said monoclonal antibody or proteolytic fragment thereof is a Fab fragment; a recombinant antibody; a recombinant Fab antibody fragment; or a recombinant single chain antibody.

32.-34. (canceled)

35. A pharmaceutical composition according to claim 30 wherein said molecule comprising the antigen-binding portion of an antibody comprises a VH-CDR3 region selected from a group consisting of polypeptides set forth in anyone of SEQ ID NOS: 1-9 and a VL-CDR3 region selected from a group consisting of polypeptides set forth in anyone of SEQ ID NOS: 10-18; a VH region selected from a group of polypeptides set forth in anyone of SEQ ID NOS: 19-27 and a VL region selected from the group of polypeptides set forth in anyone of SEQ ID NOS: 28-36; a VH-CDR3 region encoded by a polynucleotide sequence set forth in anyone of SEQ ID NOS: 39-47 and a VL-CDR3 region selected from a group consisting of polypeptides set forth in anyone of SEQ ID NOS: 48-56; or a VH region encoded by a polynucleotide sequence selected from a group of polynucleotides set forth in anyone of SEQ ID NOS: 57-65 and a VL region encoded by a polynucleotide sequence selected from the group of polynucleotides set forth in anyone of SEQ ID NOS: 66-74.

36. A pharmaceutical composition according to claim 35 wherein said molecule comprising the antigen-binding portion of an antibody which has a specific affinity for FGFR3 comprises a VH-CDR3 region set forth in SEQ ID NO: 1 and a VL-CDR3 region set forth in SEQ ID NO: 10; a VH region set forth in SEQ ID NO: 19 and a VL region set forth in SEQ ID NO: 28; a VH-CDR3 region encoded by a polynucleotide sequence set forth in SEQ ID NO: 39 and a VL-CDR3 region set forth in SEQ ID NO:48; a VH region encoded by a polynucleotide set forth in SEQ ID NO: 57 and a VL region encoded by a polynucleotide set forth in SEQ ID NO: 66; a single chain Fv encoded by a polynucleotide having a sequence set forth in SEQ ID NOS: 37 or 38; or is PEGylated.

37.-46. (canceled)

47. A pharmaceutical composition for the prevention, attenuation or treatment of a B cell malignancy comprising as an active ingredient an antibody in accordance with claim 29 in combination with at least one of an antibody or a chemotherapeutic agent.


SEQUENCE LISTING (74 sequences)

SEQ ID NO: 1 - protein, 8 aa, artificial sequence
  Synthetic Peptide
SYYPDFDY

SEQ ID NO: 2 - protein, 9 aa, artificial sequence
  Synthetic Peptide
DFLGYEFDY

SEQ ID NO: 3 - protein, 20 aa, artificial sequence
  Synthetic Peptide
YHSWYEMGYYGSTVGYMFDY

SEQ ID NO: 4 - protein, 10 aa, artificial sequence
  Synthetic Peptide
DNWFKPFSDV

SEQ ID NO: 5 - protein, 10 aa, artificial sequence
  Synthetic Peptide
VNHWTYTFDY

SEQ ID NO: 6 - protein, 17 aa, artificial sequence
  Synthetic Peptide
GYWYAYFTYINYGYFDN

SEQ ID NO: 7 - protein, 18 aa, artificial sequence
  Synthetic Peptide
TWQYSYFYYLDGGYYFDI

SEQ ID NO: 8 - protein, 17 aa, artificial sequence
  Synthetic Peptide
NMAYTNYQYVNMPHFDY

SEQ ID NO: 9 - protein, 17 aa, artificial sequence
  Synthetic Peptide
SMNSTMYWYLRRVLFDH

SEQ ID NO: 10 - protein, 9 aa, artificial sequence
  Synthetic Peptide
QSYDGPDLW

SEQ ID NO: 11 - protein, 9 aa, artificial sequence
  Synthetic Peptide
QSYDYSADY

SEQ ID NO: 12 - protein, 8 aa, artificial sequence
  Synthetic Peptide
QSYDFDFA

SEQ ID NO: 13 - protein, 8 aa, artificial sequence
  Synthetic Peptide
QQYDSIPY

SEQ ID NO: 14 - protein, 8 aa, artificial sequence
  Synthetic Peptide
QQMSNYPD

SEQ ID NO: 15 - protein, 9 aa, artificial sequence
  Synthetic Peptide
QSYDNNSDV

SEQ ID NO: 16 - protein, 8 aa, artificial sequence
  Synthetic Peptide
QQTNNAPV

SEQ ID NO: 17 - protein, 8 aa, artificial sequence
  Synthetic Peptide
QSYDYFKL

SEQ ID NO: 18 - protein, 9 aa, artificial sequence
  Synthetic Peptide
QSYDMYMYI

SEQ ID NO: 19 - protein, 120 aa, artificial sequence
  Synthetic Peptide
QVQLQQSGPGLVKPSQTLSLTCAISGDSVSSNSAAWNWIRQSPGRGLEWLGRTYYRSKWYNDYAVSVKSRITINPDTSKNQFSLQLNSVTPEDTAVYYCARSYYPDFDYWGQGTLVTVSS

SEQ ID NO: 20 - protein, 118 aa, artificial sequence
  Synthetic Peptide
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARDFLGYEFDYWGQGTLVTVSS

SEQ ID NO: 21 - protein, 130 aa, artificial sequence
  Synthetic Peptide
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARYHSWYEMGYYGSTVGYMFDYWGQGTLVTVSS

SEQ ID NO: 22 - protein, 119 aa, artificial sequence
  Synthetic Peptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDNWFKPFSDVWGQGTLVTVSS

SEQ ID NO: 23 - protein, 119 aa, artificial sequence
  Synthetic Peptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVNHWTYTFDYWGQGTLVTVSS

SEQ ID NO: 24 - protein, 126 aa, artificial sequence
  Synthetic Peptide
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARGYWYAYFTYINYGYFDNWGQGTLVTVSS

SEQ ID NO: 25 - protein, 127 aa, artificial sequence
  Synthetic Peptide
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARTWQYSYFYYLDGGYYFDIWGQGTLVTVSS

SEQ ID NO: 26 - protein, 126 aa, artificial sequence
  Synthetic Peptide
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARNMAYTNYQYVNMPHFDYWGQGTLVTVSS

SEQ ID NO: 27 - protein, 126 aa, artificial sequence
  Synthetic Peptide
QVQLVQSGAEVKKPGASVKVSCKASGYTFTSYYMHWVRQAPGQGLEWMGWINPNSGGTNYAQKFQGRVTMTRDTSISTAYMELSSLRSEDTAVYYCARSMNSTMYWYLRRVLFDHWGQGTLVTVSS

SEQ ID NO: 28 - protein, 109 aa, artificial sequence
  Synthetic Peptide
DIELTQPPSVSVAPGQTARISCSGDALGDKYASWYQQKPGQAPVLVIYDDSDRPSGIPERFSGSNSGNTATLTISGTQAEDEADYYCQSYDGPDLWVFGGGTKLTVLGQ

SEQ ID NO: 29 - protein, 109 aa, artificial sequence
  Synthetic Peptide
DIELTQPPSVSVAPGQTARISCSGDALGDKYASWYQQKPGQAPVLVIYDDSDRPSGIPERFSGSNSGNTATLTISGTQAEDEADYYCQSYDYSADYVFGGGTKLTVLGQ

SEQ ID NO: 30 - protein, 108 aa, artificial sequence
  Synthetic Peptide
DIELTQPPSVSVAPGQTARISCSGDALGDKYASWYQQKPGQAPVLVIYDDSDRPSGIPERFSGSNSGNTATLTISGTQAEDEADYYCQSYDFDFAVFGGGTKLTVLGQ

SEQ ID NO: 31 - protein, 115 aa, artificial sequence
  Synthetic Peptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYDSIPYTFGQGTKVEIKRT

SEQ ID NO: 32 - protein, 110 aa, artificial sequence
  Synthetic Peptide
DIVLTQSPATLSLSPGERATLSCRASQSVSSSYLAWYQQKPGQAPRLLIYGASSRATGVPARFSGSGSGTDFTLTISSLEPEDFATYYCQQMSNYPDTFGQGTKVEIKRT

SEQ ID NO: 33 - protein, 112 aa, artificial sequence
  Synthetic Peptide
DIALTQPASVSGSPGQSITISCTGTSSDVGGYNYVSWYQQHPGKAPKLMIYDVSNRPSGVSNRFSGSKSGNTASLTISGLQAEDEADYYCQSYDNNSDVVFGGGTKLTVLGQ

SEQ ID NO: 34 - protein, 110 aa, artificial sequence
  Synthetic Peptide
DIVLTQSPATLSLSPGERATLSCRASQSVSSSYLAWYQQKPGQAPRLLIYGASSRATGVPARFSGSGSGTDFTLTISSLEPEDFATYYCQQTNNAPVTFGQGTKVEIKRT

SEQ ID NO: 35 - protein, 108 aa, artificial sequence
  Synthetic Peptide
DIELTQPPSVSVAPGQTARISCSGDALGDKYASWYQQKPGQAPVLVIYDDSDRPSGIPERFSGSNSGNTATLTISGTQAEDEADYYCQSYDYFKLVFGGGTKLTVLGQ

SEQ ID NO: 36 - protein, 112 aa, artificial sequence
  Synthetic Peptide
DIALTQPASVSGSPGQSITISCTGTSSDVGGYNYVSWYQQHPGKAPKLMIYDVSNRPSGVSNRFSGSKSGNTASLTISGLQAEDEADYYCQSYDMYNYIVFGGGTKLTVLGQ

SEQ ID NO: 37 - protein, 249 aa, artificial sequence
  Synthetic Peptide
MXTCAISGNSVSSNSAAWNWIRQSPGRGLEWLGRTYYRSKWYNDYAVSVKSRITINPDTSKNQFSLQLNSVTPEDTAVYYCARSYYPDFDYWGQGTLVTVSSAGGGSGGGGSGGGGSGGGGSDIELTQPPSVSVAPGQTARISCSGDALGDKYASWYQQKPGQAPVLVIYDDSDRPSGIPERFSGSNSGNTATLTISGTQAEDEADYYCQSYDGPDLWVFGGGTKLTVLGQEFDYKMTMTKRAVEPPAV

SEQ ID NO: 38 - DNA, 750 bp, artificial sequence
  Synthetic Oligonucleotide
atgnnnacctgtgcgatttccgggaatagcgtgagcagcaacagcgcggcgtggaactggattcgccagtctcctgggcgtggcctcgagtggctgggccgtacctattatcgtagcaaatggtataacgattatgcggtgagcgtgaaaagccggattaccatcaacccggatacttcgaaaaaccagtttagcctgcaactgaacagcgtgaccccggaagatacggccgtgtattattgcgcgcgttcttattatcctgattttgattattggggccaaggcaccctggtgacggttagctcagcgggtggcggttctggcggcggtgggagcggtggcggtggttctggcggtggtggttccgatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgacggtcctgatctttgggtgtttggcggcggcacgaagttaaccgttcttggccaggaattcgactataagatgacgatgacaaagcgcgccgtggagccacccgcagtttga

SEQ ID NO: 39 - DNA, 24 bp, artificial sequence
  Synthetic Oligonucleotide
tcttattatcctgattttgattat

SEQ ID NO: 40 - DNA, 27 bp, artificial sequence
  Synthetic Oligonucleotide
gattttcttggttatgagtttgattat

SEQ ID NO: 41 - DNA, 60 bp, artificial sequence
  Synthetic Oligonucleotide
tatcattcttggtatgagatgggttattatggttctactgttggttatatgtttgattat

SEQ ID NO: 42 - DNA, 30 bp, artificial sequence
  Synthetic Oligonucleotide
gataattggtttaagcctttttctgatgtt

SEQ ID NO: 43 - DNA, 30 bp, artificial sequence
  Synthetic Oligonucleotide
gttaatcattggacttatacttttgattat

SEQ ID NO: 44 - DNA, 51 bp, artificial sequence
  Synthetic Oligonucleotide
ggttattggtatgcttattttacttatattaattatggttattttgataat

SEQ ID NO: 45 - DNA, 54 bp, artificial sequence
  Synthetic Oligonucleotide
acttggcagtattcttatttttattatcttgatggtggttattattttgatatt

SEQ ID NO: 46 - DNA, 51 bp, artificial sequence
  Synthetic Oligonucleotide
aatatggcttatactaattatcagtatgttaatatgcctcattttgattat

SEQ ID NO: 47 - DNA, 51 bp, artificial sequence
  Synthetic Oligonucleotide
tctatgaattctactatgtattggtatcttcgtcgtgttctttttgatcat

SEQ ID NO: 48 - DNA, 27 bp, artificial sequence
  Synthetic Oligonucleotide
cagagctatgacggtcctgatctttgg

SEQ ID NO: 49 - DNA, 27 bp, artificial sequence
  Synthetic Oligonucleotide
cagagctatgactattctgctgattat

SEQ ID NO: 50 - DNA, 24 bp, artificial sequence
  Synthetic Oligonucleotide
cagagctatgactttgattttgct

SEQ ID NO: 51 - DNA, 24 bp, artificial sequence
  Synthetic Oligonucleotide
cagcagtatgattctattccttat

SEQ ID NO: 52 - DNA, 24 bp, artificial sequence
  Synthetic Oligonucleotide
cagcagatgtctaattatcctgat

SEQ ID NO: 53 - DNA, 27 bp, artificial sequence
  Synthetic Oligonucleotide
cagagctatgacaataattctgatgtt

SEQ ID NO: 54 - DNA, 24 bp, artificial sequence
  Synthetic Oligonucleotide
cagcagactaataatgctcctgtt

SEQ ID NO: 55 - DNA, 24 bp, artificial sequence
  Synthetic Oligonucleotide
cagagctatgactattttaagctt

SEQ ID NO: 56 - DNA, 27 bp, artificial sequence
  Synthetic Oligonucleotide
cagagctatgacatgtataattatatt

SEQ ID NO: 57 - DNA, 362 bp, artificial sequence
  Synthetic Oligonucleotide
caggtgcaattgcaacagtctggtccgggcctggtgaaaccgagccaaaccctgagcctgacctgtgcgatttccggagatagcgtgagcagcaacagcgcggcgtggaactggattcgccagtctcctgggcgtggcctcgagtggctgggccgtacctattatcgtagcaaatggtataacgattatgcggtgagcgtgaaaagccggattaccatcaacccggatacttcgaaaaaccagtttagcctgcaactgaacagcgtgaccccggaagatacggccgtgtattattgcgcgcgttcttattatcctgattttgattattggggccaaggcaccctggtgacggttagctcagc

SEQ ID NO: 58 - DNA, 356 bp, artificial sequence
  Synthetic Oligonucleotide
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtgattttcttggttatgagtttgattattggggccaaggcaccctggtgacggttagctcagc

SEQ ID NO: 59 - DNA, 392 bp, artificial sequence
  Synthetic Oligonucleotide
caggtgcaattgaaagaaagcggcccggccctggtgaaaccgacccaaaccctgaccctgacctgtaccttttccggatttagcctgtccacgtctggcgttggcgtgggctggattcgccagccgcctgggaaagccctcgagtggctggctctgattgattgggatgatgataagtattatagcaccagcctgaaaacgcgtctgaccattagcaaagatacttcgaaaaatcaggtggtgctgactatgaccaacatggacccggtggatacggccacctattattgcgcgcgttatcattcttggtatgagatgggttattatggttctactgttggttatatgtttgattattggggccaaggcaccctggtgacggttagctcagc

SEQ ID NO: 60 - DNA, 359 bp, artificial sequence
  Synthetic Oligonucleotide
caggtgcaattggttcagtctggcgcggaagtgaaaaaaccgggcagcagcgtgaaagtgagctgcaaagcctccggaggcacttttagcagctatgcgattagctgggtgcgccaagcccctgggcagggtctcgagtggatgggcggcattattccgatttttggcacggcgaactacgcgcagaagtttcagggccgggtgaccattaccgcggatgaaagcaccagcaccgcgtatatggaactgagcagcctgcgtagcgaagatacggccgtgtattattgcgcgcgtgataattggtttaagcctttttctgatgtttggggccaaggcaccctggtgacggttagctcagc

SEQ ID NO: 61 - DNA, 309 bp, artificial sequence
  Synthetic Oligonucleotide
cgtgaaagtgagctgcaaagcctccggaggcacttttagcagctatgcgattagctgggtgcgccaagcccctgggcagggtctcgagtggatgggcggcattattccgatttttggcacggcgaactacgcgcagaagtttcagggccgggtgaccattaccgcggatgaaagcaccagcaccgcgtatatggaactgagcagcctgcgtagcgaagatacggccgtgtattattgcgcgcgtgttaatcattggacttatacttttgattattggggccaaggcaccctggtgacggttagctcagc

SEQ ID NO: 62 - DNA, 330 bp, artificial sequence
  Synthetic Oligonucleotide
cgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtggttattggtatgcttattttacttatattaattatggttattttgataattggggccaaggcaccctggtgacggttagctcagc

SEQ ID NO: 63 - DNA, 383 bp, artificial sequence
  Synthetic Oligonucleotide
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtacttggcagtattcttatttttattatcttgatggtggttattattttgatatttggggccaaggcaccctggtgacggttagctcagc

SEQ ID NO: 64 - DNA, 380 bp, artificial sequence
  Synthetic Oligonucleotide
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgtaatatggcttatactaattatcagtatgttaatatgcctcattttgattattggggccaaggcaccctggtgacggttagctcagc

SEQ ID NO: 65 - DNA, 380 bp, artificial sequence
  Synthetic Oligonucleotide
caggtgcaattggttcagagcggcgcggaagtgaaaaaaccgggcgcgagcgtgaaagtgagctgcaaagcctccggatatacctttaccagctattatatgcactgggtccgccaagcccctgggcagggtctcgagtggatgggctggattaacccgaatagcggcggcacgaactacgcgcagaagtttcagggccgggtgaccatgacccgtgataccagcattagcaccgcgtatatggaactgagcagcctgcgtagcgaagatacggccgtgtattattgcgcgcgttctatgaattctactatgtattggtatcttcgtcgtgttctttttgatcattggggccaaggcaccctggtgacggttagctcagc

SEQ ID NO: 66 - DNA, 327 bp, artificial sequence
  Synthetic Oligonucleotide
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgacggtcctgatctttgggtgtttggcggcggcacgaagttaaccgttcttggccag

SEQ ID NO: 67 - DNA, 327 bp, artificial sequence
  Synthetic Oligonucleotide
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactattctgctgattatgtgtttggcggcggcacgaagttaaccgttcttggccag

SEQ ID NO: 68 - DNA, 324 bp, artificial sequence
  Synthetic Oligonucleotide
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactttgattttgctgtgtttggcggcggcacgaagttaaccgttcttggccag

SEQ ID NO: 69 - DNA, 345 bp, artificial sequence
  Synthetic Oligonucleotide
gatatcgtgatgacccagagcccggatagcctggcggtgagcctgggcgaacgtgcgaccattaactgcagaagcagccagagcgtgctgtatagcagcaacaacaaaaactatctggcgtggtaccagcagaaaccaggtcagccgccgaaactattaatttattgggcatccacccgtgaaagcggggtcccggatcgttttagcggctctggatccggcactgattttaccctgaccatttcgtccctgcaagctgaagacgtggcggtgtattattgccagcagtatgattctattccttatacctttggccagggtacgaaagttgaaattaaacgtacg

SEQ ID NO: 70 - DNA, 330 bp, artificial sequence
  Synthetic Oligonucleotide
gatatcgtgctgacccagagcccggcgaccctgagcctgtctccgggcgaacgtgcgaccctgagctgcagagcgagccagagcgtgagcagcagctatctggcgtggtaccagcagaaaccaggtcaagcaccgcgtctattaatttatggcgcgagcagccgtgcaactggggtcccggcgcgttttagcggctctggatccggcacggattttaccctgaccattagcagcctggaacctgaagactttgcgacttattattgccagcagatgtctaattatcctgatacctttggccagggtacgaaagttgaaattaaacgtacg

SEQ ID NO: 71 - DNA, 336 bp, artificial sequence
  Synthetic Oligonucleotide
gatatcgcactgacccagccagcttcagtgagcggctcaccaggtcagagcattaccatctcgtgtacgggtactagcagcgatgtgggcggctataactatgtgagctggtaccagcagcatcccgggaaggcgccgaaactgatgatttatgatgtgagcaaccgtccctcaggcgtgagcaaccgttttagcggatccaaaagcggcaacaccgcgagcctgaccattagcggcctgcaagcggaagacgaagcggattattattgccagagctatgacaataattctgatgttgtgtttggcggcggcacgaagttaaccgttcttggccag

SEQ ID NO: 72 - DNA, 330 bp, artificial sequence
  Synthetic Oligonucleotide
gatatcgtgctgacccagagcccggcgaccctgagcctgtctccgggcgaacgtgcgaccctgagctgcagagcgagccagagcgtgagcagcagctatctggcgtggtaccagcagaaaccaggtcaagcaccgcgtctattaatttatggcgcgagcagccgtgcaactggggtcccggcgcgttttagcggctctggatccggcacggattttaccctgaccattagcagcctggaacctgaagactttgcgacttattattgccagcagactaataatgctcctgttacctttggccagggtacgaaagttgaaattaaacgtacg

SEQ ID NO: 73 - DNA, 324 bp, artificial sequence
  Synthetic Oligonucleotide
gatatcgaactgacccagccgccttcagtgagcgttgcaccaggtcagaccgcgcgtatctcgtgtagcggcgatgcgctgggcgataaatacgcgagctggtaccagcagaaacccgggcaggcgccagttctggtgatttatgatgattctgaccgtccctcaggcatcccggaacgctttagcggatccaacagcggcaacaccgcgaccctgaccattagcggcactcaggcggaagacgaagcggattattattgccagagctatgactattttaagcttgtgtttggcggcggcacgaagttaaccgttcttggccag

SEQ ID NO: 74 - DNA, 336 bp, artificial sequence
  Synthetic Oligonucleotide
gatatcgcactgacccagccagcttcagtgagcggctcaccaggtcagagcattaccatctcgtgtacgggtactagcagcgatgtgggcggctataactatgtgagctggtaccagcagcatcccgggaaggcgccgaaactgatgatttatgatgtgagcaaccgtccctcaggcgtgagcaaccgttttagcggatccaaaagcggcaacaccgcgagcctgaccattagcggcctgcaagcggaagacgaagcggattattattgccagagctatgacatgtataattatattgtgtttggcggcggcacgaagttaaccgttcttggccag